(19) United States
(12) Patent Application Publication
(10) Pub. No.: US 2026/0364021 A1
(43) Pub. Date: Nov. 19, 2026

(54) CORE-SHELL CAPSULES AND USES THEREOF

(71) Applicant: PRESIDENT AND FELLOWS OF HARVARD COLLEGE, Cambridge, MA (US)

(72) Inventors: Allon KLEIN, Cambridge, MA (US); Ignas MAZELIS, Cambridge, MA (US)

(73) Assignee: PRESIDENT AND FELLOWS OF HARVARD COLLEGE, Cambridge, MA (US)

(21) Appl. No.: 19/099,263

(22) PCT Filed: Aug. 3, 2023
(86) PCT No.: PCT/US2023/029364

§ 371 Date: Jan. 28, 2025

Related U.S. Application Data
(60) Provisional application No. 63/394,820, filed on Aug. 3, 2022.

(51) Int. Cl. A61K 9/50; A61K 31/711; A61K 35/36

(52) U.S. Cl. CPC A61K 9/5031; A61K 9/501; A61K 9/5015; A61K 31/711; A61K 35/36

(57) ABSTRACT

The present disclosure relates generally to core-shell capsules, methods of preparing and uses thereof.

### CROSS-REFERENCE TO RELATED APPLICATIONS

This application is a 371 National Phase Entry of International Patent Application No. PCT/US23/29364, filed Aug. 3, 2023, which claims benefit under 35 U.S.C. § 119 (e) of U.S. Provisional Application No. 63/394,820, filed Aug. 3, 2022, the contents of both of which are incorporated herein by reference in their entireties.

### GOVERNMENT SUPPORT

This invention was made with government support under CA218579, HG012771, and CA278392 awarded by National Institutes of Health (NIH). The Government has certain rights in the invention.

### TECHNICAL FIELD

The present disclosure relates generally to core-shell capsules and uses thereof.

### BACKGROUND

Capsules formed by co-flowing dextran and polyethylene glycol (PEG) in a microfluidic device are known. In these known capsules, the PEG phase and dextran phase undergo separation in which the PEG phase forms a shell around the dextran phase, which after being cross-linked, forms the core-shell capsule (i.e., a capsule having a PEG shell and a dextran core).

Unfortunately, these known dextran/core-PEG/shell capsules have several problems. First, these known capsules are highly sensitive to pH and specific density, which often results in capsules that do not have a uniform concentric core-shell structure, and which also makes it difficult to introduce changes to the crosslinking density or porosity of the capsules to suit the need of different applications without compromising capsule concentricity. Lack of a uniform concentric core-shell structure generally results in ruptured shells and/or premature release of any encapsulated material from the capsule. Second, in these dextran/core-PEG/shell capsules the PEG phase and dextran phase often do not completely separate, resulting in capsules that are not optically clear. Third, these dextran/core-PEG/shell capsules generally do not have a specific molecular weight cutoff for permeability, which makes these capsules ineffective as scaffolds for retaining compounds such as nucleic acids, proteins or drugs within the capsules. Having a sharp molecular weight cutoff is desirable, as it allows capsules to retain nucleic acids (e.g., genomic DNA, mRNA from cells, or a specific template (viral) for diagnostic purposes) while enabling reagents, such as primers and enzymes to enter and process (e.g., amplify, modify, digest) the retained nucleic acid.

Therefore, there is an ongoing need for improved core-shell capsules. Embodiments of the various aspects described herein address that need.

### SUMMARY

In one aspect provided herein is a capsule, e.g., a core shell capsule. The capsule comprises (a) a liquid core; and (b) a shell surrounding the liquid core. The shell comprises a cross-linked poly(ethylene glycol)-poly(propylene oxide)-poly(ethylene glycol) diacrylate triblock copolymer.

In some embodiments of any one of the aspects described herein, the shell is semi-permeable. In some embodiments of any one of the aspects described herein, the core comprises a solute. In some embodiments of any one of the aspects described herein, the solute is a viscosity modifier. In some embodiments of any one of the aspects described herein, the solute is a sugar. In some embodiments of any one of the aspects described herein, the solute is dextran. In some embodiments of any one of the aspects described herein, the dextran has a molecular weight in the range of 3000 Daltons to 2,000,000 Daltons. In some embodiments of any one of the aspects described herein, the capsule is a microcapsule. In some embodiments of any one of the aspects described herein, the capsule has a diameter of from about 1 μm to about 1000 μm. In some embodiments of any one of the aspects described herein, the capsule has a diameter of from about 5 μm to about 200 μm. In some embodiments of any one of the aspects described herein, the capsule is optically clear. In some embodiments of any one of the aspects described herein, the capsule further comprises a coating covering an outer surface of the shell, wherein the coating is a biocompatible material. In some embodiments of any one of the aspects described herein, the coating comprises a fluorinated compound, polyethylene glycol (PEG), poloxamer, gelatin, silanizing agent, collagen, fibrinogen, laminin, or bovine serum albumin (BSA). In some embodiments of any one of the aspects described herein, the capsule further comprises an agent. In some embodiments of any one of the aspects described herein, the agent is in the core. In some embodiments of any one of the aspects described herein, the agent is in the shell. In some embodiments of any one of the aspects described herein, the agent is a cell, biomolecule, polymer, small organic or inorganic molecule, microorganism or organoid. In some embodiments of any one of the aspects described herein, the agent is an amino acid, peptide, polypeptide, nucleotide, oligonucleotide, polynucleotide, saccharide, oligosaccharide, or polysaccharide. In some embodiments of any one of the aspects described herein, the agent is an enzyme, an antibody, a primer nucleic acid, or a plasmid. In some embodiments of any one of the aspects described herein, the agent is a cell. In some embodiments of any one of the aspects described herein, the agent is a therapeutic agent or an imaging agent. In some embodiments of any one of the aspects described herein, the capsule further comprises a high molecular weight molecule in the core. In some embodiments of any one of the aspects described herein, the high molecular weight molecule comprises at least one functional group for forming a linkage with an agent. In some embodiments of any one of the aspects described herein, the high molecular weight molecule is a high molecular weight polymer. In some embodiments of any one of the aspects described herein, the capsule further comprises a cross-linking molecule in the shell. In some embodiments of any one of the aspects described herein, the cross-linking molecule comprises at least one functional group for forming a linkage with an agent. In some embodiments of any one of the aspects described herein, the capsule further includes one or more beads in the core. In some embodiments of any one of the aspects described herein, the bead within the capsule has a cross-linking molecule on its surface. In some embodiments of any one of the aspects described herein, the cross-linking molecule on the bead in the capsule forms a linkage with an agent, such as an antibody.

In another aspect provided herein is a method for preparing a capsule. In some embodiments of any one of the aspects described herein, the method comprises: (i) forming an emulsion comprising droplets of an aqueous phase solution disposed in a non-aqueous phase solution, wherein the droplets comprise a liquid core surrounded by a shell comprising an uncross-linked polymer blend, wherein the uncross-linked polymer blend comprises an uncross-linked poly(ethylene glycol)-poly(propylene glycol)-poly(ethylene glycol) diacrylate triblock copolymer and an uncross-linked poly(ethylene glycol) diacrylate homopolymer; and (ii) cross-linking the polymer blend, and wherein the capsule comprises: (a) a liquid core; and (b) a shell surrounding the liquid core, wherein the shell comprises a cross-linked polymer blend, wherein the cross-linked polymer blend comprises a cross-linked poly(ethylene glycol)-poly(propylene glycol)-poly(ethylene glycol) diacrylate triblock copolymer and cross-linked poly(ethylene glycol) diacrylate homopolymer.

It is noted that the amount of the homopolymer, or the length of the homopolymer, or the length of the triblock copolymer, or the relative length of the different blocks of the triblock copolymer in the shell can be adjusted to optimize different shell properties such as permeability. For example, the homopolymer can be omitted from the shell. Accordingly, in another aspect, provided herein is a method for preparing a capsule, where the shell does not comprise a cross-linked homopolymer. The method comprises: (i) forming an emulsion comprising droplets of an aqueous phase solution disposed in a non-aqueous phase solution, wherein the droplets comprise a liquid core surrounded by a shell comprising a poly(ethylene glycol)-poly(propylene glycol)-poly(ethylene glycol) diacrylate triblock copolymer; and (ii) cross-linking the copolymer, and wherein the capsule comprises: (a) a liquid core; and (b) a cross-linked shell surrounding the liquid core, wherein the cross-linked shell comprises a cross-linked poly(ethylene glycol)-poly(propylene glycol)-poly(ethylene glycol) triblock copolymer.

In one example, a single aqueous phase comprising diacrylate triblock copolymer (e.g., Pluronic® F127 DA) and dextran is mixed with a fluorinated oil to form droplets. The droplets are heated to induce micelle formation thereby initiating phase separation of dextran and diacrylate triblock copolymer (e.g., Pluronic® F127 DA) into a core:shell capsule inside the droplets.

It is noted that cross-linking can be initiated chemically or by light. Accordingly, in some embodiments of any one of the aspects described herein, the step of cross-linking comprises exposing the emulsion to ultraviolet (UV) radiation or visible light radiation. In some other embodiments, the step of cross-linking comprises adding a chemical cross-linking initiator to the emulsion.

In some embodiments of the various aspects, the step of forming the emulsion comprises mixing the aqueous phase solution with the uncross-linked copolymer or the uncross-linked polymer blend to form a first mixture and mixing the first mixture with the non-aqueous phase solution. In some embodiments of any one of the aspects described herein, the step of forming the emulsion is in a microfluidic device. In some embodiments of any one of the aspects described herein, wherein the step of forming the emulsion comprises: feeding the aqueous phase solution into a first microfluidic channel of a microfluidic device; feeding the uncross-linked copolymer or the uncross-linked polymer blend into a second microfluidic channel of the microfluidic device; feeding the non-aqueous phase solution into a fourth microfluidic channel of the microfluidic device; mixing the aqueous phase solution with the uncross-linked copolymer or the uncross-linked polymer blend at a first intersection between the first microfluidic channel and the second microfluidic channel to form a first mixture; conveying the first mixture through a third microfluidic channel toward a second intersection between the third microfluidic channel and the fourth microfluidic channel; mixing the first mixture with the non-aqueous phase solution at the second intersection to form the emulsion; and collecting the emulsion from the microfluidic device.

In some embodiments, the method further comprises adding a cross-linking initiator, e.g., a photoinitiator or a chemical cross-linking initiator to the emulsion prior to the step of cross-linking. In some embodiments, the non-aqueous phase solution comprises an oil, e.g., a fluorinated oil such as a fluorine-substituted alkylsiloxane or a fluorocarbon or a hydrofluoroether. In some embodiments, the method further comprises coating an outer surface of the cross-linked shell, e.g., with a biocompatible material. In some embodiments, the method further comprises adding an agent to the capsule. In some embodiments, the method further comprises adding a high molecular weight molecule in the liquid core. In some embodiments, the method further comprises adding a cross-linking molecule in the shell, e.g., a cross-linking molecule comprising at least one functional group for forming a linkage with an agent.

Embodiments of the various aspects described herein include a homopolymer in the shell. When the homopolymer is included in the shell, an amount of the homopolymer in the shell can be higher or lower relative to an amount of the triblock copolymer (w/w or v/v). Generally, a ratio of homopolymer to triblock copolymer in the shell is from about 99:1 to about 1:99 (w/w or v/v). For example, the ratio of homopolymer to triblock copolymer in the shell is from about 0.5:1 to about 1:50 (w/w or v/v). In some embodiments of any one of the aspects described herein, the ratio of homopolymer to triblock copolymer is from about 0.5:1 to about 1:25 (w/w or v/v). In some embodiments of any one of the aspects described herein, the ratio of homopolymer to triblock copolymer in the shell is from about 1:2 to about 1:20 (w/w or v/v). For example, the ratio of homopolymer to triblock copolymer in the shell is from about 1:2 to about 1:20, from about 1:2 to about 1:8, from about 1:8 to about 4:8, from about 1:8 to about 1:16, from about 1:2 to about 1:8, from about 0.5:8 to about 1:8, from about 4:18 to about 1:10, or from about 4:10 to about 1:18 (w/w or v/v).

The capsules described herein are suitable for various uses. For example, the capsules described herein can be used in single-cell genomics, immune-isolation, cytometry, co-culture screens, micro-environmental screens, protein evolution, enzymatic cascade reactions, drug screening, cell cultures, parallel clonal expansion of single cells, isolation of DNA and RNA, enzymatic processing of DNA or RNA including but not limited to ligation, reverse transcription, transposition and polymerase chain reaction (PCR), imaging, drug delivery, and/or diagnostics.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C depict, in accordance with various embodiments of the invention, schematic diagrams illustrating properties of non-limiting examples of capsules of the invention.

FIGS. 2A-2D depict, in accordance with various embodiments of the invention, schematic of method of capsule preparation (FIG. 2A), and micrographs showing capsules in 1×PBS buffer (FIG. 2B-FIG. 2D). Arrows show single cells (FIG. 2D). Scale bar=50 μm (FIG. 2C-FIG. 2D).

FIG. 3 depicts various embodiments of the invention.

FIGS. 4A-4C depict, various embodiments of the invention with varying levels of dextran input with stable capsule diameter quantified in FIG. 4B and resulting change in resulting shell thickness, quantified in FIG. 4C.

FIG. 5. 5A-5B depict, various embodiments of the invention showing assay design (FIG. 5A) and results (FIG. 5B) showing different diffusion times through the 70 μm 8% F127:11% dextran capsule shell in DPBS for different molecules. Diffusion speed can be modulated with crosslinker-addition of 1% PEG4DA during capsule production increases the diffusion times.

FIGS. 6A-6B depict, in accordance with various embodiments of the invention, the diffusion times through the capsule shell for a single analyte (a DNA oligonucleotide) using the assay shown in FIG. 5A. The diffusion times are measured through the capsule shell for different capsule synthesis conditions and buffer conditions varying dextran concentration during synthesis, detergent concentration in the buffer during diffusion (FIGS. 6A and 6B) and temperature of the capsules during diffusion (FIG. 6A).

FIGS. 7A-7B depict, in accordance with various embodiments of the invention, an assay design and results to show PCR amplification of DNA in a capsule and subsequent retention of PCR amplified product (FIG. 7A), and the dissolution of capsules and recovery of DNA from capsules (FIG. 7B).

FIGS. 12A-12B depict, in accordance with various embodiments of the invention, flow cytometry of capsules containing diffuse (FIG. 12A) or concentrated signal (FIG. 12B).

FIG. 13 depicts, various embodiments of the invention, a schematic for hydrogel capsules to be used as compartments containing cell derived material for high-throughput multi-modal readouts.

FIGS. 14A-14C depicts, in accordance with various embodiments of the invention, an assay and results for multi-step processing of capsules for whole-transcriptome analysis. Selectively retained mRNAs derived from single-cells lysed in capsules are captured in a form of cDNA by in-capsule reverse-transcription (RT) reaction followed by PCR amplification. Images show 8% F127:11% dextran capsules containing single-cell derived and amplified cDNA stained with SybrGreen DNA dye (FIG. 14A). Cells are seen as puncta owing to gentle lysis in this example while empty capsules show no signal. Single-cell derived cDNA can be purified from hydrogel capsules (FIG. 14B) and can be used for RNA sequencing library construction (FIG. 14C).

FIG. 15 depict, in accordance with various embodiments of the invention, results comparing gene-expression data between RNA sequencing protocols carried out using cell in solution (bulk) and 8% F127:11% dextran capsules of the invention.

FIGS. 16A-16B depict, in accordance with various embodiments of the invention, single cell multi-omic workflow with capsules.

FIG. 17 depicts, in accordance with various embodiments of the invention, a method for nucleic acid barcoding in capsules. DNA can be barcoded in the capsules by combinatorically adding a short unique nucleic acid sequence in a stepwise manner. Several steps of sequence addition results in capsules housing individually unique sequence.

FIGS. 18A-18C depict, in accordance with various embodiments of the invention, a workflow and results for single-cell RNA sequencing using capsules of the invention. Selectively retained mRNAs derived from single-cells lysed in capsules is captured in a form of cDNA by in-capsule reverse-transcription (RT) reaction. Amplified cDNA is barcoded inside capsules by doing 3 rounds of unique sequence additions, followed by barcoded cDNA purification, RNA-seq library preparation and next generation sequencing (FIG. 18A). Combinatorial barcoding of cDNA in capsules accurately tag individual capsules (FIG. 18B) and enables quantification of single-cell gene-expression (FIG. 18C, D).

FIGS. 19A-19B depicts, in accordance with various embodiments of the invention, a workflow and results for single-cell RNA sequencing of a mixture of human and mouse cells using capsules of the invention. Multi-step processing and barcoding of mixed species cells in capsules (FIG. 19A) captures the clean transcriptomes of individuals cells without cross-contamination between capsules (FIG. 19B).

FIGS. 20A-20B depict, in accordance with various embodiments of the invention, a workflow and results for chromatin accessibility assay using capsules of the invention. The accessibility of selectively retained native chromatin of individually encapsulated and lysed cells is captured using Tn5 tagmentation. Resulting gDNA fragments are amplified inside capsules using PCR, purified, and sequenced using next generation sequencing (FIG. 20A). Accessibility assay done on isolated nuclei in solution and cell in F127 capsules show the same transcription start site (TSS) enrichment score (FIG. 20B)

FIGS. 21A-21B depict, in accordance with various embodiments of the invention, a workflow (FIG. 21A) and results (FIG. 21B) for single-cell derived material preservation in the capsules by freezing at −80 °C.

FIG. 22 depicts, in accordance with various embodiments of the invention, non-limiting strategy for a high-throughput sequencing-free cytometry of single-cells or cell colonies for, but not limited to, gene-expression, growth, perturbation response.

FIG. 23 depicts, in accordance with various embodiments of the invention, a non-limiting strategy for a high-throughput combinatorial drug or perturbation screening on cells growing in the capsules.

FIG. 24 depicts, in accordance with various embodiments of the invention, non-limiting strategy for a preparation of therapeutic cells or cargo material which will be physically isolated from surrounding tissue upon introduction into relevant tissue or host.

FIG. 25 depicts, in accordance with various embodiments of the invention, non-limiting strategy for mammalian cell-cell co-culture, where one cell is a ‘reporter’ that responds to a signal exuded by another cell from a library, such as, but not limited to, antibody producing cell, T cell, CAR-T cell and enables the selection or enrichment of a specific phenotype. Illustrative image of a single Pluronic® F127 capsule housing cell of two types interacting.

FIG. 26 depicts, in accordance with various embodiments of the invention, non-limiting strategies for high-throughput screening libraries of antibodies for a specific binding target.

FIG. 27 depicts, in accordance with various embodiments of the invention, a non-limiting strategy for using capsules to house series of enzymes for enzymatic cascade reactions.

FIG. 28 depicts, in accordance with various embodiments of the invention, increasing Pluronic® F127 concentration facilitates PEGDA capsule formation.

FIG. 29 depicts, in accordance with various embodiments of the invention, addition of Pluronic® F127 stabilizes the formation of PEGDA shell capsules.

FIGS. 30A-30B depict, in accordance with various embodiments of the invention, non-limiting strategy for immobilizing small proteins inside capsules of the invention.

FIG. 31 depicts in accordance with various embodiments of the invention, capsules are permissive of clonal cell growth.

### DETAILED DESCRIPTION

Without being bound by theory, phase separation between the core phase and shell phase is key to forming the core-shell capsule. The core-shell capsules described herein provide several surprising benefits and improvements over the known dextran/core-PEG/shell capsules. First, the use of one or more poloxamers (e.g., various poly(ethylene glycol)-poly(propylene oxide)-poly(ethylene glycol) triblock copolymers) in combination with one or more acrylate functionalized PEG homopolymers in the preparation of core:shell capsules unexpectedly stabilized the capsules. Second, replacement of the one or more acrylate functionalized PEG homopolymers with one or more acrylate functionalized poloxamers (e.g., a poly(ethylene glycol)-poly(propylene oxide)-poly(ethylene glycol) diacrylate triblock copolymer) provide capsules that have significantly improved properties over the known dextran/core-PEG/shell capsules, including without limitation, higher pore size, and increased control over permeability of the shell by tuning the poloxamer length and concentration.

The inventors have discovered inter alia that a key parameter of core-shell capsule formation is the ability of the acrylate functionalized poloxamer(s) to aggregate into a gel-like solution or micelle(s). Formation of this gel-like solution or micelle provides stable core:shell capsules comprising a liquid core and a shell comprising cross-linked polymer(s). Accordingly, in one aspect, provided herein is a capsule comprising: (a) a liquid core; and (b) a shell surrounding the liquid core. The shell comprises a cross-linked poly(ethylene glycol)-poly(propylene oxide)-poly(ethylene glycol) diacrylate triblock copolymer.

Embodiments of the various aspects described herein include a poly(ethylene glycol)-poly(propylene oxide)-poly(ethylene glycol) diacrylate triblock copolymer. Generally, the poly(ethylene glycol)-poly(propylene glycol)-poly(ethylene glycol) diacrylate triblock copolymer comprises a structure of Formula I:

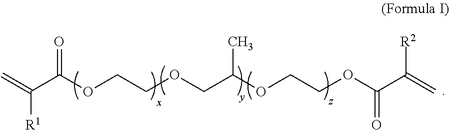

In polymers of Formula I, x is 1-200, y is 1-200, z is 1-200, and R¹ and R² independently are H or C₁-C₆alkyl (e.g., methyl).

Poly(ethylene glycol)-poly(propylene oxide)-poly(ethylene glycol) triblock copolymers are also referred to as poloxamers in the art and commonly known by the trade names Pluronic®, Kolliphor®, and Synperonic®. Thus, in some embodiments of any one of the aspects described herein, the triblock copolymer is a diacrylate poloxamer. For example, the triblock copolymer is a diacrylated Pluronic®.

In some embodiments of any one of the aspects described herein, the uncross-linked poly(ethylene glycol)-poly(propylene glycol)-poly(ethylene glycol) diacrylate triblock copolymer has a molecular weight (M_{w}) of about 1,000 Daltons to about 20,000 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 2,000 Daltons to about 19,000 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 3,000 Daltons to about 18,000 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 4,000 Daltons to about 17,000 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 5,000 Daltons to about 15,000 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 14,600 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 12,500 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 8,400 Daltons. In some embodiments of any one of the aspects described herein, the uncross-linked poly(ethylene glycol)-poly(propylene glycol)-poly(ethylene glycol) diacrylate triblock copolymer has a molecular weight (M_{w}) of about 5,800 Daltons. Non-limiting examples of uncross-linked poly(ethylene glycol)-poly(propylene glycol)-poly(ethylene glycol) triblock copolymers include (poly(ethylene glycol)₁₀₀-poly(propylene glycol)₆₅-poly(ethylene glycol)₁₀₀ diacrylate (i.e., Pluronic® F127 DA), (poly(ethylene glycol)₇₆-poly(propylene glycol)₂₉-poly(ethylene glycol)₇₆ diacrylate (i.e., Pluronic® F68 DA), (poly(ethylene glycol)₁₃₆-poly(propylene glycol)₅₂-poly(ethylene glycol)₁₃₆ diacrylate (i.e., Pluronic® F108 DA), and poly(ethylene glycol)₂₀-poly(propylene glycol)₆₉-poly(ethylene glycol)₂₀ diacrylate (i.e., Pluronic® P123 DA).

In some embodiments of any one of the aspects described herein, the shell further comprises a homopolymer, e.g., a cross-linked homopolymer. For example, the shell further comprises a poly(ethylene glycol) diacrylate homopolymer, e.g., a cross-linked poly(ethylene glycol) diacrylate homopolymer. In some embodiments of any one of the aspects described herein, the shell is substantially free of homopolymers. For example, the shell is substantially free of poly(ethylene glycol) diacrylate or polyethylene glycol.

Generally, the uncross-linked poly(ethylene glycol) diacrylate homopolymer comprises a structure of Formula II:

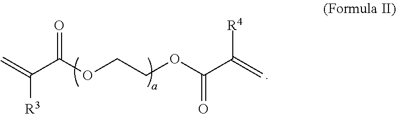

In homopolymers of Formula II, a is 1-10,000 and R³ and R⁴ independently are H or C₁-C₆alkyl (e.g., methyl).

Non-limiting examples of the uncross-linked poly(ethylene glycol) diacrylate homopolymer include poly(ethylene glycol)₄ diacrylate (PEG4-DA), poly(ethylene glycol)₅₇₅ diacrylate (PEG575-DA), poly(ethylene glycol)₇₅₀ diacrylate (PEG750-DA), and poly(ethylene glycol)₂₀₀₀ diacrylate (PEG2000-DA).

In some embodiment of any one of the aspects described herein, a ratio of homopolymer to triblock copolymer is from about 99:1 to about 1:99 (w/w or v/v). In some embodiment of the various aspects described herein, a ratio of homopolymer to triblock copolymer is from about 1:2 (homopolymer:triblock copolymer) to about 1:8 (homopolymer:triblock copolymer) (w/w or v/v). In some embodiments of any one of the aspects described herein, a ratio of homopolymer to triblock copolymer is from about 1:8 (homopolymer:triblock copolymer) to about 1:16 (homopolymer:triblock copolymer) (w/w or v/v).

In some embodiment of any one of the aspects described herein, an amount of the homopolymer is higher relative to an amount of the triblock copolymer (w to w or v to v). In some embodiment of any one of the aspects described herein, an amount of the homopolymer to an amount of the triblock copolymer is about 10%-18% homopolymer to about 1%-4% triblock copolymer.

In some embodiment of any one of the aspects described herein, an amount of the homopolymer to an amount of the triblock copolymer is about 0.5%-1% homopolymer to about 8% triblock copolymer. In some embodiment of any one of the aspects described herein, an amount of the homopolymer to an amount of the triblock copolymer is about 1%-4% homopolymer to about 8% triblock copolymer.

In some embodiment of any one of the aspects described herein, the shell is semi-permeable. As used herein, “semi-permeable” means permeable to the passage of some molecules but not all, e.g., a selective impediment to the passage of fluids and/or substances in the fluids. Semi-permeability is one of the key functions that substantially increases the usability of capsules. In some embodiments of any one of the aspects described herein, the semi-permeable shell prevents the passage of macromolecules and cells, but allows the passage of smaller molecules. In some embodiments of any one of the aspects described herein, the passage of one or more biologically active molecules is allowed. In some embodiments of any one of the aspects described herein, the semi-permeability is adjusted by changing the poly(ethylene glycol)-poly(propylene oxide)-poly(ethylene glycol) diacrylate triblock copolymer or poly(ethylene glycol) diacrylate monomers length.

For example, the capsule has a diameter of about 2 μm to about 500 μm. In some embodiments of any one of the aspects described herein, the capsule has a diameter of about 3 μm to about 300 μm. For example, the capsule has a diameter of about 4 μm to about 250 μm. In some embodiments of any one of the aspects described herein, the capsule has a diameter of about 5 μm to about 200 μm.

One skilled in the art will understand that the capsules generally show a size distribution around the indicated “diameter”. Unless otherwise indicated, the terms “capsule diameter” as used herein refer to the mode of size distribution of capsules, i.e., the value that appears most frequently in the size distribution. Methods for measuring capsule or particle size are known to those skilled in the art, for example, by visible light microscopy and fluorescence microscopy (for capsules of diameter 1-1000 μm), and (for capsules <1 μm diameter) by dynamic light scattering (such as photocorrelation spectroscopy, laser diffraction, low angle laser light scattering (LALLS) and medium angle laser light scattering (MALLS)), light obscuration procedures (such as the Coulter analysis procedure), or other techniques (such as rheology and light or electron microscopy).

It is noted that the capsule described herein can be of spherical or non-spherical shape. In some embodiments of any one of the aspects described herein, the capsule can be substantially spherical. What is meant by “substantially spherical” is that the ratio of the lengths of the longest to the shortest perpendicular axes of the cross-section of the capsule is less than or equal to about 1.5. Substantially spherical does not require a line of symmetry. In addition, capsules can have surface texturing, such as lines or indentations or bumps that are small in scale compared to the total size of the capsule and still be substantially spherical. In some embodiments of any one of the aspects described herein, the ratio of lengths between the longest and shortest axes of the capsule is less than or equal to about 1.5, less than or equal to about 1.45, less than or equal to about 1.4, less or equal to about 1.35, less than or equal to about 1.30, less than or equal to about 1.25, less than or equal to about 1.20, less than or equal to about 1.15 less than or equal to about 1, one. Without wishing to be bound by theory, surface contact is minimized in capsules that they are substantially spherical, which minimizes unwanted agglomeration of capsules during storage. Many crystals or flakes have flat surfaces that can allow large surface contact areas where agglomeration can take place by ionic or non-ionic interactions. A sphere allows contact in a much smaller area.

In some embodiments of any one of the aspects described herein, the capsules have substantially the same particle size. Capsules that have a broad size distribution in which relatively large and small capsules are found allow smaller capsules to fill the gaps between capsules, thus creating new contact surfaces. A wide size distribution can result in larger spheres by creating many contact opportunities for the joining of an agglomeration. The capsules described herein are within a narrow size distribution, thus minimizing the opportunities for contact agglomeration. What is meant by a “narrow size distribution” is a particle size distribution that has a relationship between the 90th percentile volumetric diameter of small spherical particles and the 10th percentile volumetric diameter less than or equal to 5. In some forms For realization, the volumetric diameter of the 90th percentile of small spherical particles with respect to the volumetric diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal 1.3, less than or equal to 1.25, less than or equal to 1.20, less than or equal to 1.15, or less than or equal to 1.1.

The geometric standard deviation (GSD) can also be used to indicate the narrow size distribution. The GSD calculations involved determining the effective cutting diameter (ECD) in the cumulative percentages less than 15.9% and 84.1%. The GSD is equal to the square root of the ratio of ECD less than 84.17% to ECD less than 15.9%. The GSD has a narrow size distribution when GSD<2.5. In some embodiments of any one of the aspects described herein, the GSD is less than 2, less than 1.75, or less than 1.5. In one embodiment, GSD is less than 1.8.

In some embodiments of any one of the aspects described herein, the capsule is optically clear.

In some embodiments of any one of the aspects described herein, the capsule further comprises a coating covering an outer surface of the shell. The coating can comprise a biocompatible material. The term “biocompatible” refers a substance that is substantially non-toxic.

In some embodiments of any one of the aspects described herein, the coating comprises a material selected from the group consisting of fluorinated compounds, polyethylene glycol (PEG), poloxamers, gelatins, silanizing agent, collagen, fibrinogen, laminin, bovine serum albumin (BSA), and any combinations thereof.

In some embodiments of any one of the aspects described herein, the capsule further comprises an agent. Exemplary agents for including in the capsules described herein include, but are not limited to, cells, biomolecules, polymers, small organic or inorganic molecules, microorganisms and organoids.

In some embodiments of any one of the aspects described herein, the agent is an amino acid, peptide, polypeptide, nucleotide, oligonucleotide, polynucleotide, saccharide, oligosaccharide, or polysaccharide.

In some embodiments of any one of the aspects described herein, the agent is a cell, biomolecule, polymer, small organic or inorganic molecule, microparticle, bead, microorganism or organoid. In some embodiments of any one of the aspects described herein, the microparticle has a diameter of at least 100 nm and at most half the diameter of the capsule. In some embodiments of any one of the aspects described herein, the microparticle comprises polystyrene, polymethacrylate, or another polymer. In some embodiments of any one of the aspects described herein, the bead has a diameter of at least 100 nm and at most half the diameter of the capsule. In some embodiments of any one of the aspects described herein, the bead comprises polystyrene, polymethacrylate, or another polymer.

In some embodiments of any one of the aspects describe herein, the agent is an enzyme, an antibody, a primer nucleic acid, or a plasmid.

In some embodiments of any one of the aspects described herein, the agent is a cell.

In some embodiments of any one of the aspects described herein, the agent is a therapeutic agent.

In some embodiments of any one of the aspects described herein, the agent is an imaging agent.

It is noted that the agent can be located in the core and/or the shell. In some embodiments of any one of the aspects described herein, the agent is in the core. In some other embodiments of any one of the aspects described herein, the agent is in the shell.

In some embodiments of any one of the aspects described herein, the agent is covalently linked with a component in the capsule. For example, the agent is covalently linked with a component in the shell. In another non-limiting example, the agent is covalently linked with a component in the core.

In some embodiments of any one of the aspects described herein, the capsule further comprises a high molecular weight molecule or a bead in the core. In some embodiments of any of the aspects described herein, the high molecular weight molecule in the core is amino dextran, chitosan, or gelatin. In some embodiments of any one of the aspects described herein, the high molecular weight molecule or the bead comprises at least one functional group for forming a linkage with an agent. In some embodiments of any one of the aspects described herein, the high molecular weight molecule is a high molecular weight polymer.

In some embodiments of any one of the aspects described herein, the capsule further comprises a cross-linking molecule in the shell. In some embodiments of any one of the aspects described herein, the cross-linking molecule comprises at least one functional group for forming a linkage with an agent.

### Methods for Preparing the Capsules

In another aspect provided herein is a method for preparing a capsule described herein. Generally, the method comprises forming an emulsion comprising droplets of an aqueous phase solution disposed in a non-aqueous phase solution. The droplets comprise a liquid core surrounded by a shell comprising uncross-linked poly(ethylene glycol)-poly(propylene glycol)-poly(ethylene glycol) diacrylate triblock copolymer. After forming the emulsion, the triblock copolymer in the shell is cross-linked to produce the capsule.

In some embodiments of any one of the aspects described herein, the shell comprises poly(ethylene glycol) diacrylate homopolymer, e.g., cross-linked poly(ethylene glycol) diacrylate homopolymer. Such capsules can be produced by the methods described herein using a mixture or blend of uncross-linked poly(ethylene glycol)-poly(propylene glycol)-poly(ethylene glycol) diacrylate triblock copolymer and uncross-linked poly(ethylene glycol) diacrylate homopolymer. Generally, the method comprises forming an emulsion comprising droplets of an aqueous phase solution disposed in a non-aqueous phase solution. The droplets comprise a liquid core surrounded by a shell comprising uncross-linked polymer blend comprising uncross-linked poly(ethylene glycol)-poly(propylene glycol)-poly(ethylene glycol) diacrylate triblock copolymer and uncross-linked poly(ethylene glycol) diacrylate homopolymer. After forming the emulsion, the polymer blend in the shell is cross-linked to produce the capsule.

In some embodiments of the any one of the aspects described herein, the uncross-linked triblock copolymer is an acrylate terminated poly(ethylene glycol)-poly(propylene glycol)-poly(ethylene glycol) triblock copolymer (e.g., an acrylate terminated poloxamer). In some embodiments of any one of the aspects described herein, the uncross-linked triblock copolymer is an acrylate functionalized poly(ethylene glycol)-poly(propylene glycol)-poly(ethylene glycol) triblock copolymer (e.g., an acrylate functionalized poloxamer). In some embodiments of any one of the aspects described herein, the uncross-linked triblock copolymer is of Formula I.

In some embodiments of any one of the aspects described herein, the uncross-linked poly(ethylene glycol) diacrylate homopolymer is of Formula II.

The step of forming the emulsion comprises mixing the aqueous phase solution with the uncross-linked triblock copolymer or the uncross-linked the polymer blend to form a first mixture and mixing the first mixture with a non-aqueous phase solution.

In some embodiments of any one of the aspects described herein, the emulsion can be formed in a microfluidic device. For example, the step of forming the emulsion comprises: feeding the aqueous phase solution into a first microfluidic channel of a microfluidic device; feeding the uncross-linked triblock copolymer or the uncross-linked the polymer blend into a second microfluidic channel of the microfluidic device; feeding the non-aqueous phase solution into a fourth microfluidic channel of the microfluidic device; mixing the aqueous phase solution with the uncross-linked triblock copolymer or the uncross-linked the polymer blend at a first intersection between the first microfluidic channel and the second microfluidic channel to form a first mixture; conveying the first mixture through a third microfluidic channel toward a second intersection between the third microfluidic channel and the fourth microfluidic channel; mixing the first mixture with the non-aqueous phase solution at the second intersection to form the emulsion; and collecting the emulsion from the microfluidic device.

In some embodiments of any one of the aspects described herein, cross-linking comprises photo-cross-linking. For example, the step of cross-linking comprises exposing the emulsion to ultraviolet (UV) radiation or visible light radiation. When photo-cross-linking is used, the emulsion can comprise a photoinitiator. It is noted the photoinitiator can be present in the aqueous and/or the non-aqueous phase solutions used for forming the emulsion or added to the emulsion after formation but prior to the cross-linking step. It is noted in some embodiments that when the non-aqueous phase of the emulsion is a hydrofluoroether (HFE), then generally a photoinitiator (hydrophobic or hydrophilic) will not dissolve into the non-aqueous phase of the emulsion. In some embodiments of any one of the aspects described herein, a hydrophobic initiator can be dissolved in acetone, which can then be dispersed into the non-aqueous fluorophilic phase of the emulsion.

Exemplary photoinitiators include, but are not limited to, benzoin methyl ether; benzoin isopropyl ether; 2,2-diethoxyacetophenone (Irgacure™ 651 photoinitiator); 2,2-dimethoxy-2-phenyl-1-phenylethanone (Esacure™ KB-1 photoinitiator); dimethoxyhydroxyacetophenone; 2-methyl-2-hydroxy propiophenone; 2-naphthalene-sulfonyl chloride; 1-phenyl-1,2-propanedione-2-(0-ethoxy-carbonyl) oxime; 2,4-diethyl thioxanthone; 2-tert-butyl thioxanthone; 2-chlorothioxanthone; 2-propoxy thioxanthone; 2-benzyl-2-dimethylamino-1-(4-morpholinophenyl) butan-1-one (Iracure 369™ photoinitiator); 2-methyl-1-[4-(methylthio)phenyl]-2-morpholino propan-2-one (Iracure 907™ photoinitiator); 2-hydroxy-4′-(2-hydroxy ethoxy)-2-methylpropiophenone (Irgacure-2959™ photoinitiator); lithium phenyl-2,4,6-trimethylbenzoylphosphinate (LAP); triethanolamine; N-vinyl caprolactam; benzophenone; benzil dimethyl ketal; diethoxyacetophenone; dibutoxyacetophenone; methyl phenyl glycoxylate; 2-ethylthioxanthone; 2-isopropylthioxanthone; phenyl 2-hydroxy-2-propyl ketone; 4-isopropylphenyl 2-hydroxy-2-propyl ketone; 4-n-dodecylphenyl 2-hydroxy-2propyl ketone; 4-(2-hydroxyethoxy)phenyl 2-hydroxy-2propyl ketone; 4-(2-acryloyloxyethoxy)phenyl 2-hydroxy-2-propyl ketone; 1-benzoylcyclohexanol; and Eosin Y.

In some embodiments of any one of the aspects described herein, the photoinitiator is a hydrophobic radical polymerization reaction photoinitiator. Exemplary hydrophobic radical polymerization reaction photoinitiators include, but are not limited to, 1-[4-(2-hydroxyethoxy)-phenyl]-2-hydroxy-2-methyl-1-propane-1-one; 1-hydroxy-cyclohexyl-phenyl-ketone; 2,2-dimethoxy-1,2-diphenylethan-1-one; 2,4,6-trimethylbenzoyl-diphenylphosphineoxide; 2-benzyl-2-dimethylamino-1-(4-morpholinophenyl)-butanone-1; 2-hydroxy-2-methyl-1-phenyl-propan-1-one; 2-methyl-1 [4-(methylthio)phenyl]-2-morpholinopropan-1-one;benzophenone; bis(2,4,6-trimethylbenzoyl)-phenylphosphineoxide; bis(2,6-dimethoxybenzoyl)-2,4,4-trimethyl-pentylphosphineoxide; bis(η-5-2,4-cyclopentadien-1-yl)-bis(2,6-difluoro-3-(1H-pyrrol-1-yl)-phenyl) titanium; and Irgacure® 184

In some embodiments of any one of the aspects described herein, the cross-linking comprises chemical cross-linking. For example, the step of cross-linking comprises adding a cross-linking initiator to the emulsion. Exemplary cross-linking initiators include, but are not limited to, 1,1-di-tert-butylperoxy-3,3,5-trimethylcyclohexane; 1,1-di-tert-butyl peroxycyclohexane; tert-butyl peroxy-3,3,5-trimethyl hexanoate; tert-butyl peroxy isopropylcarbonate; 2,2-di-tert-butyl peroxybutane; tert-butylperoxyacetate; 4,4-di-tert-butylperoxy-n-butyl valerate; 2,5-di-methyl-2,5-bis(benzoyl peroxide) hexane; tert-butyl peroxybenzoate; di-tert-butyl diperoxy phthalate; 2-tert-butyl-2-cyanopropane; 2-tert-butyl-1-cyanocyclohexane; O,O-tert-butyl-O-isopropyl monoperoxy carbonate; tert-butyl peroxy maleic acid; and 2,5-dimethyl-2,5-bis(octanoyl peroxy) hexane. In some embodiments of any one of the aspects described herein, the chemical cross-linking comprises adding ammonium persulfate (APS) and tetramethylethylenediamine (TEMED) to the emulsion. For example, TEMED may be dissolved in acetone and added to the non-aqueous phase of the emulsion.

The non-aqueous phase may serve as a carrier fluid forming a continuous phase that is immiscible with water, or the non-aqueous phase may be a dispersed phase. The non-aqueous phase may be referred to as an oil phase comprising at least one oil, but may include any liquid (or liquefiable) compound or mixture of liquid compounds that is immiscible with water. The oil may be synthetic or naturally occurring. The oil may or may not include carbon and/or silicon, and may or may not include hydrogen and/or fluorine. The oil may be lipophilic or lipophobic. In other words, the oil may be generally miscible or immiscible with organic solvents. Exemplary oils may include at least one silicone oil, mineral oil, fluorocarbon oil, hydrofluoroether, vegetable oil, or a combination thereof, among others

In some embodiments of any one of the aspects described herein, the non-aqueous phase solution comprises an oil. For example, non-aqueous phase solution comprises a fluorinated oil. In some embodiments of the any one of the aspects described herein, the fluorinated oil is a fluorine-substituted alkylsiloxane or a fluorocarbon or a hydrofluoroether (e.g., 3M™ Novec™ HFE-7500).

In some embodiments of any one of the aspects described herein, the method further comprises coating an outer surface of the cross-linked shell with a biocompatible material. In some embodiments of any one of the aspects described herein, the biocompatible material is a fluorinated compound, polyethylene glycol (PEG), poloxamer, gelatin, or bovine serum albumin (BSA).

In some embodiments of any one of the aspects described herein, the method further comprises adding an agent to the capsule. In some embodiments of any one of the aspects described herein, the agent is in the liquid core. In some embodiments of any one of the aspects described herein, the agent is in the cross-linked shell. In some embodiments of any one of the aspects described herein, the agent is a bead. In some embodiments of any one of the aspects described herein, the agent is a cell, biomolecule, polymer, small organic or inorganic molecule, microorganism or organoid. In some embodiments of any one of the aspects described herein, the agent is an amino acid, peptide, polypeptide, nucleotide, oligonucleotide, polynucleotide, saccharide, oligosaccharide, or polysaccharide. In some embodiments of any one of the aspects described herein, the agent is an enzyme, an antibody, a primer nucleic acid, or a plasmid. In some embodiments of any one of the aspects described herein, the agent is a cell. In some embodiments of any one of the aspects described herein, the agent is a therapeutic agent or an imaging agent. In some embodiments of any one of the aspects described herein, a plurality of agents are added simultaneously. For example, more than one cell may be added to each capsule.

In some embodiments of any one of the aspects described herein, the method further comprises adding a high molecular weight molecule in the liquid core. In some embodiments of any one of the aspects described herein, the high molecular weight molecule comprises at least one functional group for forming a linkage with an agent. In some embodiments of any one of the aspects described herein, the high molecular weight molecule is a high molecular weight polymer.

In some embodiments of any one of the aspects described herein, the method further comprises adding a cross-linking molecule in the shell. In some embodiments of any one of the aspects described herein, the cross-linking molecule comprises at least one functional group for forming a linkage with an agent.

### Uses of the Capsules

The capsules described herein are suitable for various uses. For example, the capsules described herein can be used in single-cell genomics, immune-isolation, cytometry, co-culture screens, micro-environmental screens, protein evolution, enzymatic cascade reactions, drug screening, cell cultures, nucleic acid amplification by reverse transcription and/or polymerase chain reaction (PCR), imaging, drug delivery, and/or diagnostics.

Accordingly, in various embodiments, the disclosure provides a capsule described herein for use in co-culture screening. In one example, a suspension of plasma cells from an immunized host are mixed with a reporter cell line, and then captured in capsules such that at least 50% of capsules (and most likely >95% of capsules) have at least one reporter cell encapsulated (and most likely >5 reporter cells), and each capsule has an average of 0.1-10 plasma cells. The capsules are then incubated to allow the plasma cell to produce an antibody that either has no activity on the reporter cells, or else it agonizes or antagonizes activity of the reporter cells. The capsules are then sorted based on said activity of the reporter cells, so that plasma cells eliciting the desired change in reporter cell activity are enriched. The plasma cells are either recovered, or else they are lysed and their antibody heavy chain and light chain are analyzed by sequencing.

In various embodiments, the disclosure provides a capsule described herein for use in immune-isolation. In one example, a suspension of cells or organoids are captured in capsules. The capsules are then engrafted into a patient, or into an animal.

In various embodiments, the disclosure provides a capsule described herein for use in single-cell genomics. For example, the capsule technology described here provides a platform to capture and process multiple analytes from single cells. In one example, a suspension of cells is put into capsules, so that only a single cell is present in each capsule. The cells are then lysed inside the capsules using a cell lysis buffer. Due to the semi-permeability of the capsule's shell, genomic DNA, mRNAs (and other, longer RNAs), and macro-molecules are retained. The nucleic acid retained in the capsules can then be processed by split-and-pool barcoding prior to sequencing. The nucleic acid retained in the capsules can processed using genomic protocols that include, but are not limited to single-cell RNA sequencing, single-cell genome sequencing, single cell chromatin accessibility, single-cell genome methylation, single-cell histone modification profiling, single-cell transcription binding assays, and single-cell ribosome profiling. In one example of single cell genomics, single-cells are lysed inside the capsules using strong lysis conditions and gDNA and mRNA are retained inside. Capsules are washed, followed by gDNA digestion using DNAse I treatment. Capsules are washed again and mixed with poly-T reverse-transcription (RT) primer housing a unique molecular identified (UMI). The capsules are heated to denature the secondary structures of mRNA and placed on ice to anneal the poly-T RT primers. Reverse transcription reaction mix is added to the capsules and cDNA is synthesized. After the reaction, capsules are washed several times and cDNA inside the capsules is amplified using PCR amplification. After the PCR, capsules remain intact and can be imaged to assess the number of capsules housing cellular cDNA. The volume of capsules is picked based on desired number of cells (for example, 1,000-10,000,000 cells). Unique DNA sequences are added to each of the cDNA molecule inside the capsules (molecules inside the capsules have the same sequence added, however, different from other capsules) by performing 2-5 rounds of combinatorial split-pool ligation or PCR. Briefly, capsules are distributed to different microwells housing unique adapter DNA sequences. These sequences are appended to the ends of cDNA molecules. All the capsules are pooled into a single tube, washed, and the process is repeated. At the end, each capsule has a unique sequence appended to its molecules which informs that these molecules came from the same cell. The barcoded cDNA is purified from the capsules and DNA sequencing libraries are prepared.

In various embodiments, the disclosure provides a capsule described herein for use in single-cell analysis of protein abundance. In one example, a suspension of cells is put into capsules, so that only a single cell is present in each capsule. The cells are then lysed inside the capsules using a cell lysis buffer. Due to the semi-permeability of the capsule's shell, small proteins, metabolites, and small molecules are liberated from single-cells can move freely through the capsules shell. These analytes can be crosslinked to the capsule shell or high-molecular weight polymer inside the capsule's core. The identity of the proteins can then be identified by sequencing using antibody-DNA conjugates, or by fluorescent cytometry using antibody-fluorophore probes, or else evaluated for function using fluorometric assays of enzymatic activity.

In various embodiments, the disclosure provides a capsule described herein for use in RNA and protein cytometry. In one example, a suspension of cells is put into capsules, and then processed as for single cell genomics and for analysis of protein abundance. The capsules are then allowed to form a monolayer on a slide or a coverslip suitable for mounting for imaging on microscope. A set of fluorescent-labeled probes are added and allowed to diffuse into the capsules. The probes may include but are not limited to oligonucleotides, antibodies, and aptamers. In various embodiments, the capsules are immobilized onto the slide or coverslip for serial imaging.

In various embodiments, the disclosure provides a capsule described herein for use in cell culture. In one example, a suspension of cells are put into capsules, such that each capsule contains an average of at most 0.1 cells or organoids. The cells are then allowed to grow in the capsules, such that the result cells in each capsule are of clonal origin. The capsules may be grown in the presence of other cells that condition the cell culture media.

In various embodiments, the disclosure provides a capsule described herein for use in co-culture screen.

In various embodiments, the disclosure provides a capsule described herein for use in micro-environmental screen.

In various embodiments, the disclosure provides a capsule described herein for use in protein evolution.

In various embodiments, the present invention provides a capsule for use in enzymatic cascade reaction.

In various embodiments, the disclosure provides a capsule described herein for use in drug screening.

In various embodiments, the disclosure provides a capsule described herein for use in polymerase chain reaction (PCR).

In various embodiments, the disclosure provides a capsule described herein for use in imaging.

In various embodiments, the disclosure provides a capsule described herein for use in drug delivery.

In various embodiments, the disclosure provides a capsule described herein for use in diagnostics.

Some embodiments of the present invention can be defined as any of the following numbered embodiments:
Embodiment 1: A capsule comprising: (a) a liquid core; and (b) a shell surrounding the liquid core, wherein the shell comprises a cross-linked poly(ethylene glycol)-poly(propylene oxide)-poly(ethylene glycol) diacrylate triblock copolymer.
Embodiment 2: The capsule of paragraph 1, wherein the shell is semi-permeable.
Embodiment 3: The capsule of paragraph 1 or 2, wherein the core comprises a solute.
Embodiment 4: The capsule of paragraph 3, wherein the solute is a viscosity modifier.
Embodiment 5: The capsule of paragraph 3 or 4, wherein the solute is a sugar.
Embodiment 6: The capsule of any one of paragraphs 3-5, wherein the solute is dextran.
Embodiment 7: The capsule of paragraph 6, wherein the dextran has a molecular weight in the range of 3000 Daltons to 2,000,000 Daltons.
Embodiment 8: The capsule of any one of paragraphs 1-7, wherein the capsule is a microcapsule.
Embodiment 9: The capsule of any one of paragraphs 1-8, wherein the capsule has a diameter of from about 1 μm to about 1000 μm.
Embodiment 10: The capsule of any one of paragraphs 1-8, wherein the capsule has a diameter of 5 μm to 200 μm.
Embodiment 11: The capsule of any one of paragraphs 1-10, wherein the capsule is optically clear.
Embodiment 12: The capsule of any one of paragraphs 1-11, further comprising a coating covering an outer surface of the shell, wherein the coating is a biocompatible material.
Embodiment 13: The capsule of paragraph 12, wherein the biocompatible material is a fluorinated compound, polyethylene glycol (PEG), poloxamer, gelatin, silanizing agent, collagen, fibrinogen, laminin or bovine serum albumin (BSA).
Embodiment 14: The capsule of any one of paragraphs 1-13, further comprising an agent.
Embodiment 15: The capsule of paragraph 14, wherein the agent is in the core.
Embodiment 16: The capsule of paragraph 14, wherein the agent is in the shell.
Embodiment 17: The capsule of any one of paragraphs 14-16, wherein the agent is a cell, biomolecule, polymer, small organic or inorganic molecule, microorganism or organoid.
Embodiment 18: The capsule of any one of paragraphs 14-16, wherein the agent is an amino acid, peptide, polypeptide, nucleotide, oligonucleotide, polynucleotide, saccharide, oligosaccharide, or polysaccharide.
Embodiment 19: The capsule of any one of paragraphs 14-16, wherein the agent is an enzyme, an antibody, a primer nucleic acid, or a plasmid.
Embodiment 20: The capsule of any one of paragraphs 14-16, wherein the agent is a cell.
Embodiment 21: The capsule of any one of paragraphs 14-16, wherein the agent is a therapeutic agent or an imaging agent.
Embodiment 22: The capsule of any one of paragraphs 1-21, further comprising a high molecular weight molecule in the core.
Embodiment 23: The capsule of paragraph 22, wherein the high molecular weight molecule comprises at least one functional group for forming a linkage with an agent.
Embodiment 24: The capsule of paragraph 22 or 23, wherein the high molecular weight molecule is a high molecular weight polymer.
Embodiment 25: The capsule of any one of paragraphs 1-24, further comprising a cross-linking molecule in the shell.
Embodiment 26: The capsule of paragraph 25, wherein the cross-linking molecule comprises at least one functional group for forming a linkage with an agent.
Embodiment 27: A method for preparing a capsule, the method comprising: (i) forming an emulsion comprising droplets of an aqueous phase solution disposed in a non-aqueous phase solution, wherein the droplets comprise a liquid core surrounded by a shell comprising an uncross-linked polymer blend, wherein the uncross-linked polymer blend comprises an uncross-linked poly(ethylene glycol)-poly(propylene glycol)-poly(ethylene glycol) diacrylate triblock copolymer and an uncross-linked poly(ethylene glycol) diacrylate homopolymer; and (ii) cross-linking the polymer blend, and wherein the capsule comprises: (a) a liquid core; and (b) a shell surrounding the liquid core, wherein the shell comprises a cross-linked polymer blend, wherein the cross-linked polymer blend comprises a cross-linked poly(ethylene glycol)-poly(propylene glycol)-poly(ethylene glycol) diacrylate triblock copolymer and cross-linked poly(ethylene glycol) diacrylate homopolymer.
Embodiment 28: The method of paragraph 27, wherein a ratio of homopolymer to triblock copolymer is from about Embodiment 99:1 to about Embodiment 1:99 (w/w or v/v).
Embodiment 29: The method of paragraph 27 or 28, wherein an amount of the homopolymer is higher relative to an amount of the triblock copolymer (w/w or v/v).
Embodiment 30: The method of any one of paragraphs 27-29, wherein the step of forming the emulsion comprises mixing the aqueous phase solution with the uncross-linked polymer blend to form a first mixture and mixing the first mixture with a non-aqueous phase solution.
Embodiment 31: The method of any one of paragraphs 27-30, wherein the step of forming the emulsion is in a microfluidic device.
Embodiment 32: The method of any one of paragraphs 27-29, wherein the step of forming the emulsion comprises: feeding the aqueous phase solution into a first microfluidic channel of a microfluidic device; feeding the un-crosslinked polymer blend into a second microfluidic channel of the microfluidic device; feeding the non-aqueous phase solution into a fourth microfluidic channel of the microfluidic device; mixing the aqueous phase solution with the uncross-linked polymer blend at a first intersection between the first microfluidic channel and the second microfluidic channel to form a first mixture; conveying the first mixture through a third microfluidic channel toward a second intersection between the third microfluidic channel and the fourth microfluidic channel; mixing the first mixture with the non-aqueous phase solution at the second intersection to form the emulsion; and collecting the emulsion from the microfluidic device.
Embodiment 33: The method of any one of paragraphs 27-32, wherein the cross-linking comprises photo-cross-linking.
Embodiment 34: The method of any one of paragraphs 27-33, wherein said cross-linking comprises exposing the emulsion to ultraviolet (UV) radiation or visible light radiation.
Embodiment 35: The method of any one of paragraphs 27-34, wherein the emulsion comprises a photoinitiator.
Embodiment 36: The method of paragraph 35, further comprising adding the photoinitiator to the emulsion prior to the step of cross-linking.
Embodiment 37: The method of paragraph 35 or 36, wherein the non-aqueous phase solution comprises a photoinitator.
Embodiment 38: The method of any one of paragraphs 35-37, wherein the photoinitiator is a hydrophobic radical polymerization reaction photoinitiator.
Embodiment 39: The method of any one of paragraphs 27-32, wherein the cross-linking comprises chemical cross-linking.
Embodiment 40: The method of any one of paragraphs 27-39, wherein the non-aqueous phase solution comprises an oil.
Embodiment 41: The method of paragraph 40, wherein the oil is a fluorinated oil.
Embodiment 42: The method of paragraph 41, wherein the fluorinated oil is a fluorine-substituted alkylsiloxane or a fluorocarbon.
Embodiment 43: The method of any one of paragraphs 27-42, wherein the cross-linked shell is semi-permeable.
Embodiment 44: The method of any one of paragraphs 27-43, wherein the liquid core comprises a solute.
Embodiment 45: The method of paragraph 44, wherein the solute is a viscosity modifier.
Embodiment 46: The method of paragraph 44, wherein the solute is a sugar.
Embodiment 47: The method of paragraph 44, wherein the solute is dextran.
Embodiment 48: The method of paragraph 47, wherein the dextran has a molecular weight in the range of 3000 Daltons to 2,000,000 Daltons.
Embodiment 49: The method of any one of paragraphs 27-48, wherein the capsule is a microcapsule.
Embodiment 50: The method of any one of paragraphs 27-49, wherein the capsule has a diameter of from about 1 μm to about 1000 μm.
Embodiment 51: The method of any one of paragraphs 27-49, wherein the capsule has a diameter of from about 5 μm to about 200 μm.
Embodiment 52: The method of any one of paragraphs 27-51, wherein the capsule is optically clear.
Embodiment 53: The method of any one of paragraphs 27-52, further comprising coating an outer surface of the cross-linked shell with a biocompatible material.
Embodiment 54: The method of any one of paragraphs 27-53, further comprising adding an agent to the capsule.
Embodiment 55: The method of paragraph 54, wherein the agent is in the liquid core.
Embodiment 56: The method of paragraph 54, wherein the agent is in the cross-linked shell.
Embodiment 57: The method of any one of paragraphs 54-56, wherein the agent is a cell, biomolecule, polymer, small organic or inorganic molecule, microorganism or organoid.
Embodiment 58: The method of any one of paragraphs 54-56, wherein the agent is an amino acid, peptide, polypeptide, nucleotide, oligonucleotide, polynucleotide, saccharide, oligosaccharide, or polysaccharide.
Embodiment 59: The method of any one of paragraphs 54-56, wherein the agent is an enzyme, an antibody, a primer nucleic acid, or a plasmid.
Embodiment 60: The method of any one of paragraphs 54-56, wherein the agent is a cell.
Embodiment 61: The method of any one of paragraphs 54-56, wherein the agent is a therapeutic agent or an imaging agent.
Embodiment 62: The method of any one of paragraphs 27-61, further comprising adding a high molecular weight molecule in the liquid core.
Embodiment 63: The method of paragraph 62, wherein the high molecular weight molecule comprises at least one functional group for forming a linkage with an agent.
Embodiment 64: The method of paragraph 62 or 63, wherein the high molecular weight molecule is a high molecular weight polymer.
Embodiment 65: The method of any one of paragraphs 27-64, further comprising adding a cross-linking molecule in the shell.
Embodiment 66: The capsule of paragraph 65, wherein the cross-linking molecule comprises at least one functional group for forming a linkage with an agent.
Embodiment 67: A method for preparing a capsule, the method comprising: (i) forming an emulsion comprising droplets of an aqueous phase solution disposed in a non-aqueous phase solution, wherein the droplets comprise a liquid core surrounded by a shell comprising a poly(ethylene glycol)-poly(propylene glycol)-poly(ethylene glycol) diacrylate triblock copolymer; and (ii) cross-linking the copolymer, and wherein the capsule comprises: (a) a liquid core; and (b) a cross-linked shell surrounding the liquid core, wherein the cross-linked shell comprises a cross-linked poly(ethylene glycol)-poly(propylene glycol)-poly(ethylene glycol) triblock copolymer.
Embodiment 68: The method of paragraph 67, wherein the step of forming the emulsion comprises mixing the aqueous phase solution with the copolymer to form a first mixture and mixing the first mixture with the non-aqueous phase solution.
Embodiment 69: The method of paragraph 67 or 68, wherein the step of forming the emulsion is in a microfluidic device.
Embodiment 70: The method of paragraph 67, wherein the step of forming the emulsion comprises: feeding the aqueous phase solution into a first microfluidic channel of a microfluidic device; feeding the copolymer into a second microfluidic channel of the microfluidic device; feeding the non-aqueous phase solution into a fourth microfluidic channel of the microfluidic device; mixing the aqueous phase solution with the copolymer at a first intersection between the first microfluidic channel and the second microfluidic channel to form a first mixture; conveying the first mixture through a third microfluidic channel toward a second intersection between the third microfluidic channel and the fourth microfluidic channel; mixing the first mixture with the non-aqueous phase solution at the second intersection to form the emulsion; and collecting the emulsion from the microfluidic device.
Embodiment 71: The method of any one of paragraphs 67-70, wherein the cross-linking comprises photo-cross-linking.
Embodiment 72: The method of any one of paragraphs 67-71, wherein the cross-linking comprises exposing the emulsion to ultraviolet (UV) radiation or visible light radiation.
Embodiment 73: The method of any one of paragraphs 67-72, wherein the emulsion comprises a photoinitiator.
Embodiment 74: The method of paragraph 73, further comprising adding the photoinitiator to the emulsion prior to the step of cross-linking.
Embodiment 75: The method of paragraph 73 or 74, wherein the non-aqueous phase solution comprises a photoinitator.
Embodiment 76: The method of any one of paragraphs 73-75, wherein the photoinitiator is a hydrophobic radical polymerization reaction photoinitiator.
Embodiment 77: The method of any one of paragraphs 67-70, wherein the cross-linking comprises chemical cross-linking.
Embodiment 78: The method of any one of paragraphs 67-77, wherein the non-aqueous phase solution comprises an oil.
Embodiment 79: The method of paragraphs 78, wherein the oil is a fluorinated oil.
Embodiment 80: The method of paragraph 79, wherein the fluorinated oil is a fluorine-substituted alkylsiloxane or a fluorocarbon.
Embodiment 81: The method of any one of paragraph 67-80, wherein the cross-linked shell is semi-permeable.
Embodiment 82: The method of any one of paragraphs 67-81, wherein the liquid core comprises a solute.
Embodiment 83: The method of paragraph 82, wherein the solute is a viscosity modifier.
Embodiment 84: The method of paragraph 82, wherein the solute is a sugar.
Embodiment 85: The method of paragraph 82, wherein the solute is dextran.
Embodiment 86: The method of paragraph 85, wherein the dextran has a molecular weight in the range of 3000 Daltons to 2,000,000 Daltons.
Embodiment 87: The method of any one of paragraphs 67-86, wherein the capsule is a microcapsule.
Embodiment 88: The method of any one of paragraphs 67-87, wherein the capsule has a diameter of from about 1 μm to about 1000 μm.
Embodiment 89: The method of any one of paragraphs 67-87, wherein the capsule has a diameter of from about 5 μm to 200 about μm.
Embodiment 90: The method of any one of paragraphs 67-89, wherein the capsule is optically clear.
Embodiment 91: The method of any one of paragraphs 67-90, further comprising coating an outer surface of the cross-linked shell with a biocompatible material.
Embodiment 92: The method of any one of paragraphs 67-91, further comprising adding an agent to the capsule.
Embodiment 93: The method of paragraph 92, wherein the agent is in the liquid core.
Embodiment 94: The method of paragraph 92, wherein the agent is in the cross-linked shell.
Embodiment 95: The method of any one of paragraphs 92-94, wherein the agent is a cell, biomolecule, polymer, small organic or inorganic molecule, microorganism or organoid.
Embodiment 96: The method of any one of paragraphs 92-94, wherein the agent is an amino acid, peptide, polypeptide, nucleotide, oligonucleotide, polynucleotide, saccharide, oligosaccharide, or polysaccharide:
Embodiment 97: The method of any one of paragraphs 92-94, wherein the agent is an enzyme, an antibody, a primer nucleic acid, or a plasmid.
Embodiment 98: The method of any one of paragraphs 92-94, wherein the agent is a cell.
Embodiment 99: The method of any one of paragraphs 92-94, wherein the agent is a therapeutic agent or an imaging agent.
Embodiment 100: The method of any one of paragraphs 67-99, further comprising adding a high molecular weight molecule to the liquid core.
Embodiment 101: The method of paragraph 100, wherein the high molecular weight molecule comprises at least one functional group for forming a linkage with an agent.
Embodiment 102: The method of paragraph 100 or 101, wherein the high molecular weight molecule is a high molecular weight polymer.
Embodiment 103: The method of any one of paragraphs 67-102, further comprising adding a cross-linking molecule in the shell.
Embodiment 104: The method of paragraph 103, wherein the cross-linking molecule comprises at least one functional group for forming a linkage with an agent.
Embodiment 105: Use of a capsule of any one of paragraphs 1-26 for single-cell genomics.
Embodiment 106: Use of a capsule of any one of paragraphs 1-26 for immuno-isolation.
Embodiment 107: Use of a capsule of any one of paragraphs 1-26 for cytometry.
Embodiment 108: Use of a capsule of any one of paragraphs 1-26 for co-culture screen.
Embodiment 109: Use of a capsule of any one of paragraphs 1-26 for micro-environmental screen.
Embodiment 110: Use of a capsule of any one of paragraphs 1-26 for protein evolution.
Embodiment 111: Use of a capsule of any one of paragraphs 1-26 for enzymatic cascade reaction.
Embodiment 112: Use of a capsule of any one of paragraphs 1-26 for drug screening
Embodiment 113: Use of a capsule of any one of paragraphs 1-26 for cell culture.
Embodiment 114: Use of a capsule of any one of paragraphs 1-26 for polymerase chain reaction (PCR).
Embodiment 115: Use of a capsule of any one of paragraphs 1-26 for imaging.
Embodiment 116: Use of a capsule of any one of paragraphs 1-26 for drug delivery.
Embodiment 117: Use of a capsule of any one of paragraphs 1-26 for diagnostic.
Embodiment 118: The capsule of any one of paragraphs 14-16, wherein the agent is a microparticle or bead.
Embodiment 119: The capsule of paragraph 118, wherein the microparticle has a diameter of at least 100 nm.
Embodiment 120: The capsule of paragraph 118, wherein the microparticle has a diameter that is half the diameter of the capsule.
Embodiment 121: The capsule of paragraph 118, wherein the microparticle comprises a polymer.
Embodiment 122: The capsule of paragraph 118, wherein the microparticle comprises polystyrene or polymethacrylate.
Embodiment 123: The capsule of paragraph 118, wherein the bead has a diameter of at least 100 nm.
Embodiment 124: The capsule of paragraph 118, wherein the bead has a diameter that is half the diameter of the capsule.
Embodiment 125: The capsule of paragraph 118, wherein the bead comprises a polymer.
Embodiment 126: The capsule of paragraph 118, wherein the bead comprises
polystyrene or polymethacrylate.
Embodiment 127: The capsule of any one of paragraphs 1-22, further comprising a microparticle in the core.
Embodiment 128: The capsule of paragraph 127, wherein the microparticle comprises at least one functional group for forming a linkage with an agent.
Embodiment 129: The capsule of paragraph 127 or paragraph 128, wherein the microparticle is covalently linked or non-covalently linked to an antibody.
Embodiment 130: The capsule of paragraph 22 or paragraph 23, wherein the high molecular weight molecule is amino dextran, chitosan, or gelatin.
Embodiment 131: The capsule of paragraph 22 or paragraph 23, wherein the high molecular weight molecule is covalently linked or non-covalently linked to an antibody.
Embodiment 132: The method of paragraph 38, wherein the photoinitiator is dissolved in an organic solvent.
Embodiment 133: The method of paragraph 132, wherein the organic solvent containing the photoinitiator is dispersed into the non-aqueous phase.
Embodiment 134: The method of paragraph 133, wherein the non-aqueous phase comprises a hydrofluoroether oil.
Embodiment 135: The method of paragraph 132, wherein the organic solvent is hexane or acetone.
Embodiment 136: The method of paragraph 67, further comprising heating the emulsion to induce gelation of the shell phase before cross-linking the copolymer.
Embodiment 137: The method of paragraph 136, wherein the emulsion is heated to 20-50 °C.
Embodiment 138: The method of paragraph 136, wherein the emulsion is heated to 28 °C.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

### Some Selected Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In some embodiments of any one of the aspects described herein, the numbers expressing quantities of reagents, properties such as concentration, reaction conditions, and so forth, used to describe and claim certain embodiments of the invention are to be understood as being modified in some instances by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. Accordingly, in some embodiments of any one of the aspects described herein, the numerical parameters set forth in the written description and attached claims are approximations that can vary depending upon the desired properties sought to be obtained by a particular embodiment. In some embodiments of any one of the aspects described herein, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

This study relates to a composition of matter and methods for the preparation of semi-permeable hydrogel micro-capsules with tunable properties and procedures for massively parallelized molecular biology and cell screening assays. In the following, we design and characterize micro-compartments compatible with cell growth, multi-step reactions, flow cytometry and imaging analysis. Polymerizable Pluronics act as a foundation for stable shell-core co-centric hydrogel capsule formation via simple co-flow microfluidic droplet formation followed by light induced crosslinking. The resulting Pluronic shell provides the compartment with structural integrity and acts as a size selective barrier. We define and characterize a tunable capsule composition for the selective retention of nucleic acids and rapid transport enzymes and small molecules. We showcase the use of said capsules for cell culturing, single-cell transcriptomic and genomic analysis. Millions of capsules can be analyzed in a single tube due to tuned permeability properties. The present invention establishes the foundation for high-throughput hydrogel micro-capsule based analysis and screening of cells and compounds.

### Methods, Results, and Discussion

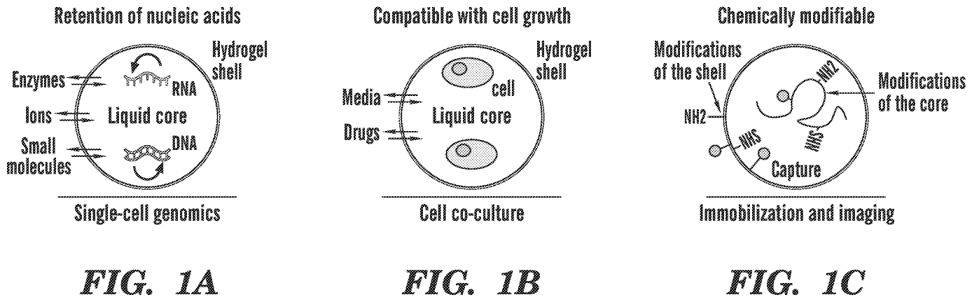

In the current state-of-the-art we define a composition of matter and an approach for the synthesis of semi-permeable hydrogel micro-capsules. With this work we established a new paradigm for massively parallelized molecular biology and cell screening assays [FIG. 1]. In the following, we design and characterize micro-compartments compatible with cell growth, multi-step reactions, flow cytometry and imaging analysis.

### Synthesis of Capsules Using Pluronic® Block Copolymer

### Methods of Synthesis of Hydrogel Capsules

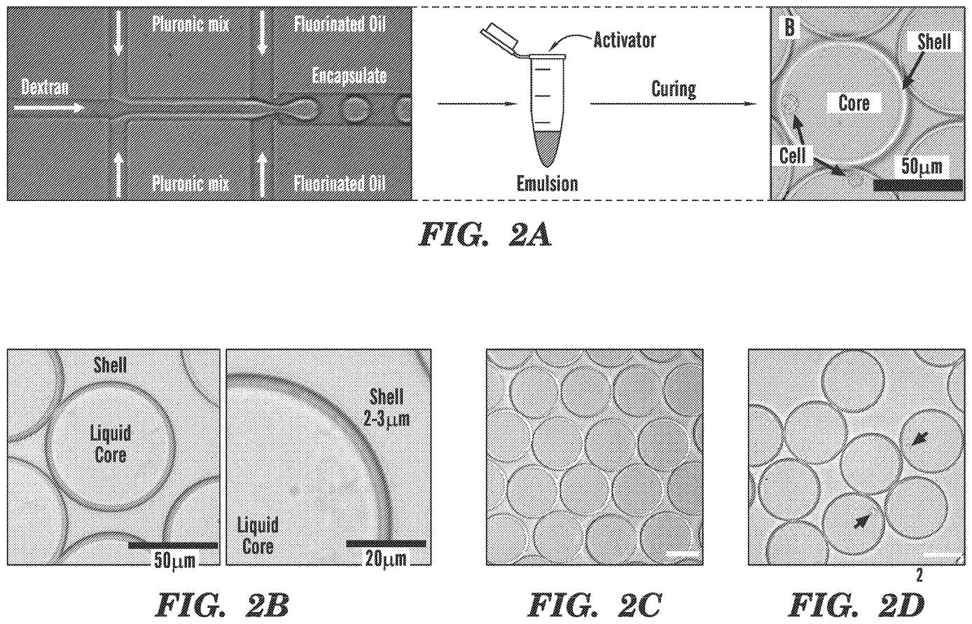

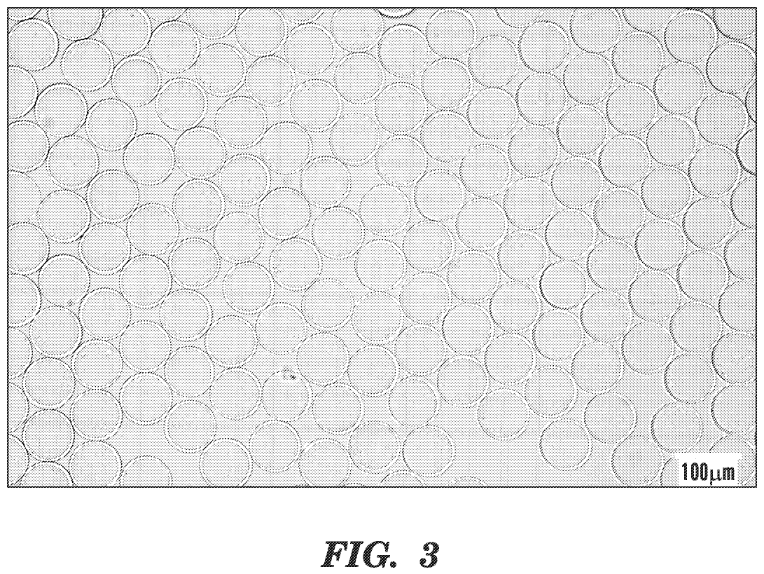

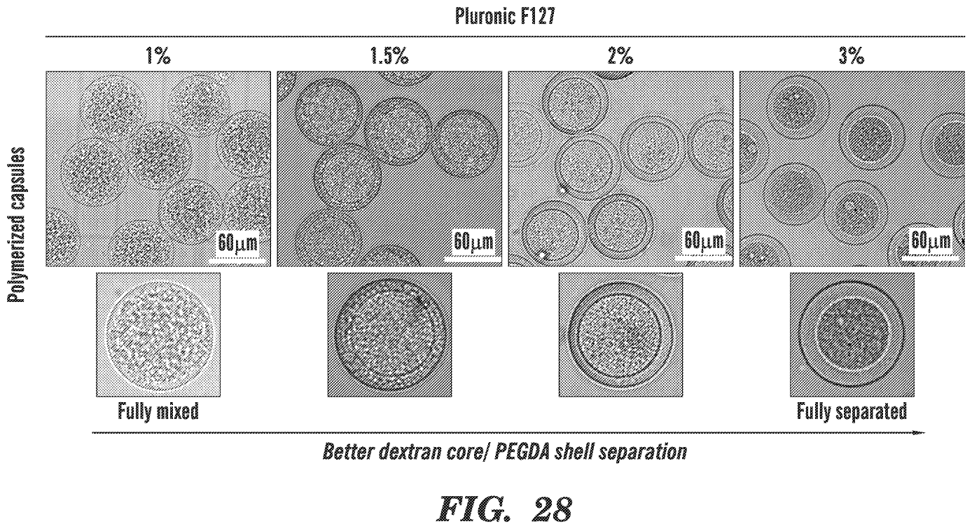

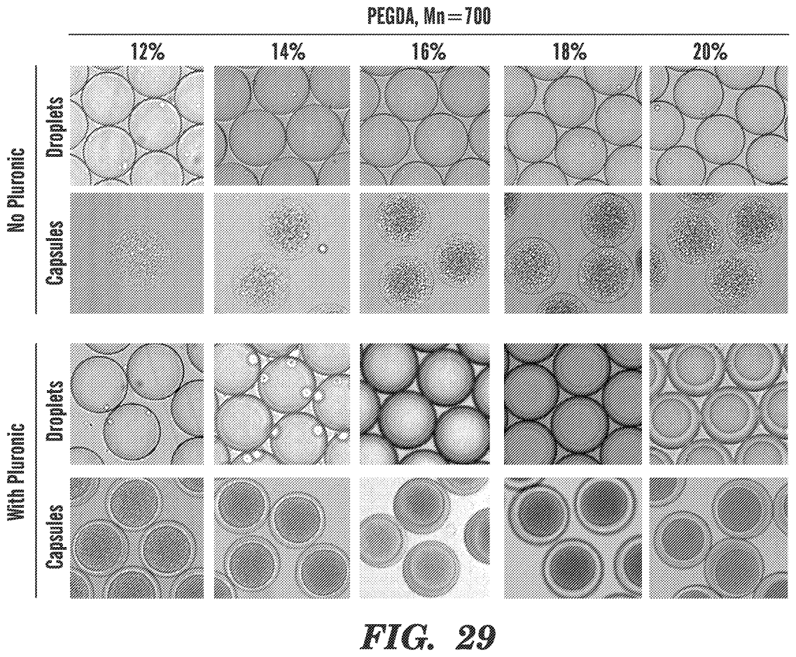

We have identified that chemically polymerizable Pluronic® block copolymers (e.g., Pluronic F127 diacrylate (F127DA)) (hereafter referred to collectively as Pluronics) can be solely used as a material to robustly form uniform, clear thin shelled hydrogel capsules. In one such example, 11% (w/w) Dextran (MW 500 kDa) in DPBS and 8% (w/w) F127DA in DPBS were formed into ~80 μm diameter droplets at 1:1 ratio for 20 mins using a 60 μm height microfluidic co-flow flow-focusing device with 2.5% fluorosurfactant in HFE7500 as a carried phase. After encapsulation, the emulsion (~200 μL) was stored on ice for 5 mins, followed by 30 °C for 3 mins. BAPO Photoinitiator was added on to the emulsion, followed by 5 s shaking and incubation at 30 °C for 3 mins. Capsules were polymerized by exposing the emulsion to 405 nm light for 5 mins. Capsules were released from the emulsion by shaking with 20% Perfluoro-octanol in HFE7500, removing oil and washing once with Hexane. After the extraction, capsules were washed several times with DPBS with 0.1% (v/v) Igepal CA-630 [FIGS. 2A-3]. F127DA hydrogel micro-capsules are robust to physical (shear force, temperature) and chemical stress and are compatible with enzymatic reactions. In addition, F127DA capsules act as base composition of matter for hydrogel capsule formation. Addition of shorter/longer Pluronics or PEG variants can be included in capsule formation without jeopardizing the stability of phase separation and enables the synthesis of semi-preamble compartments with variable diffusion characteristics. In addition, we have identified that the addition of Pluronics (etc. Pluronic F127) yields in stabilized PEG-like and Dextran polymer-polymer phase separation [FIG. 28]. This results in formation of uniform and concentric capsules with an outer PEG-rich shell phase and inner Dextran-rich core phase in variety of PEGDA and dextran concentrations [FIG. 29].

Modifications to Methods of Synthesis of Hydrogel Capsules with Varied Composition and their Diffusion Characteristics

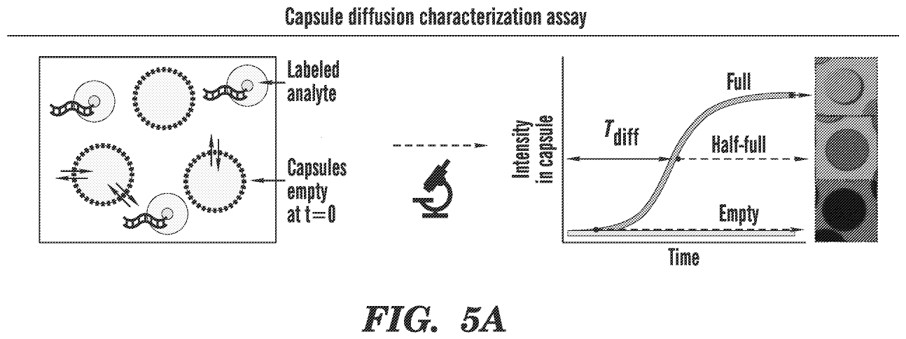

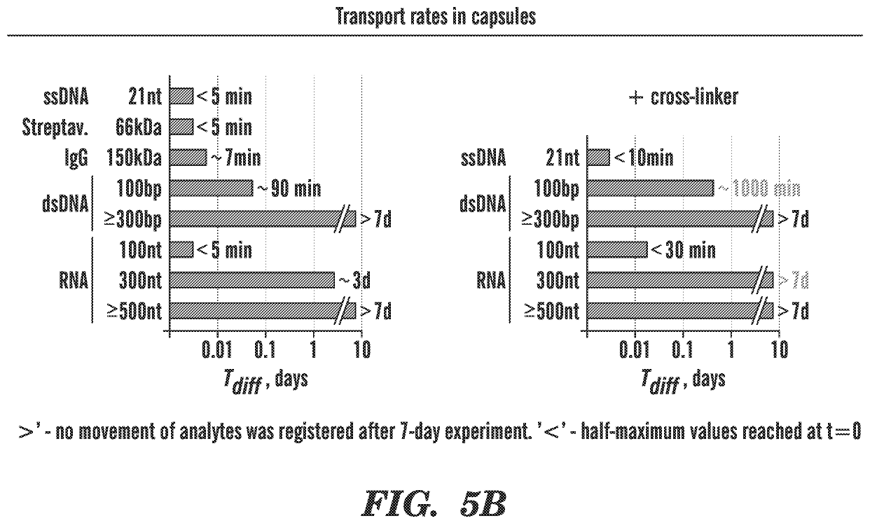

Semi-permeability of capsules should enable sequential chemical and enzymatic reactions to be performed without the loss of analytes stored inside. However, the analytes of interest might change between different applications of hydrogel microcapsules. As a result, we developed a robust platform for semi-permeable hydrogel capsules formation to enable the composition of shell (and thus the size of hydrogel pores) to be adjusted without the loss of capsule co-centricity during their synthesis. This is showcased by synthesizing capsules of varied composition and measuring the analyte transport rates thought the capsules shell. The transport characteristics were analyzed by adding empty capsules to a DPBS buffer containing the fluorescently labeled biomolecules (for example, 76 nt Cy5 labeled oligonucleotide) [FIGS. 5A and 5B]. The solution is then imaged for 1-7 day to measure fluorescence signal accumulation within the core of capsules. The resulting time series are used to infer the diffusion time through the capsule membrane. The results are displayed as time needed to reach half of the outside intensity.

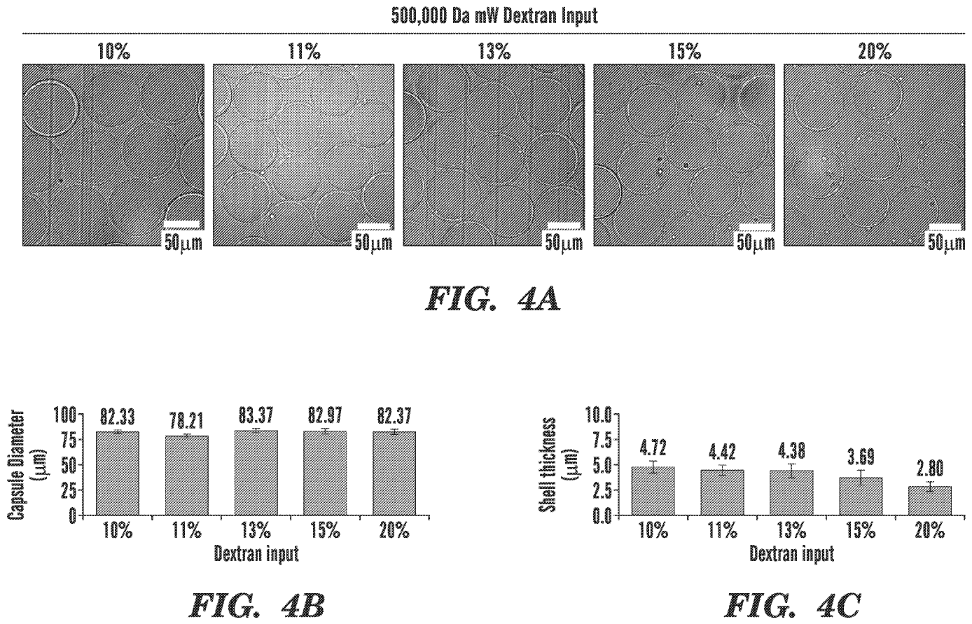

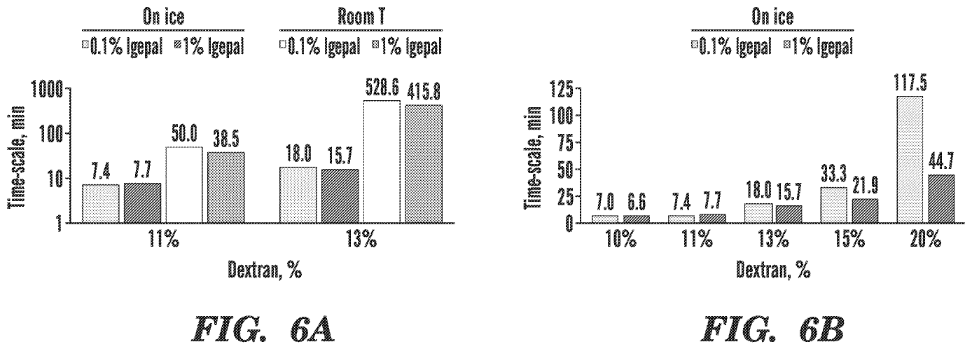

We describe several ways to control the diffusion characteristics of capsules shell. One way is to use short length Pluronics or PEGDA can be added into the shell mix and act as a crosslinker [FIG. 5A]. For example, capsules synthesized by flowing 8% F127DA in DPBS (shell mix) and 11% Dextran in DPBS (MW=500 kDa) (core mix) into microfluidic droplets show rapid diffusion of protein, RNA and DNA shorter than 300 nt, while longer molecules are retained. Addition of 1% PEGADA into shell mix results in significantly slower transport of 100 bp dsDNA and 300 nt ssDNA. Diffusion can be further constrained by increasing the concentration of short PEGDA or Pluronic monomers in the core mix. Another way to modulate the permeability of hydrogel capsules is to change the concentration of dextran in the core solution during the microfluidic encapsulation [FIGS. 4A-4C]. The higher the dextran concentration, the more the hydrogel shell gets concentrated resulting in co-centric capsules with smaller pore sizes (as inferred by slower 78 nt ssDNA primer diffusion into the capsules). To test this, hydrogel capsules were synthesized by flowing 8% F127DA, 1% PEG2000DA in DPBS and varied % of Dextran (MW=500 kDa) in DPBS into microfluidic droplets, followed by capsule polymerization. Going from 10% Dextran (MW=500 k Da) to 20% Dextran core solution, the measured shell thickness changes from 4.7 μm to 2.8 μm [FIG. 4C], resulting in 16-fold slower oligo diffusion as measured on ice in TI wash buffer (10 mM Tris 8 pH with 0.1% Igepal CA-630) [FIG. 6B]. To modulate the diffusion speed after capsule synthesis, the ambient solution properties can be altered. For example, the use of detergents (such as Triton X-100, Igepal CA-630, SDS) or lowering temperature increases the diffusion speed, as measured by the transport rate of 76 nt Cy5 labeled ssDNA oligonucleotide into empty hydrogel capsules in DPBS buffer in the presence of Igepal CA-640 or different ambient temperatures [FIGS. 6A and 6B].

### Enzymatic Reactions in Hydrogel Capsules

DNA Encapsulation into Hydrogel Capsules

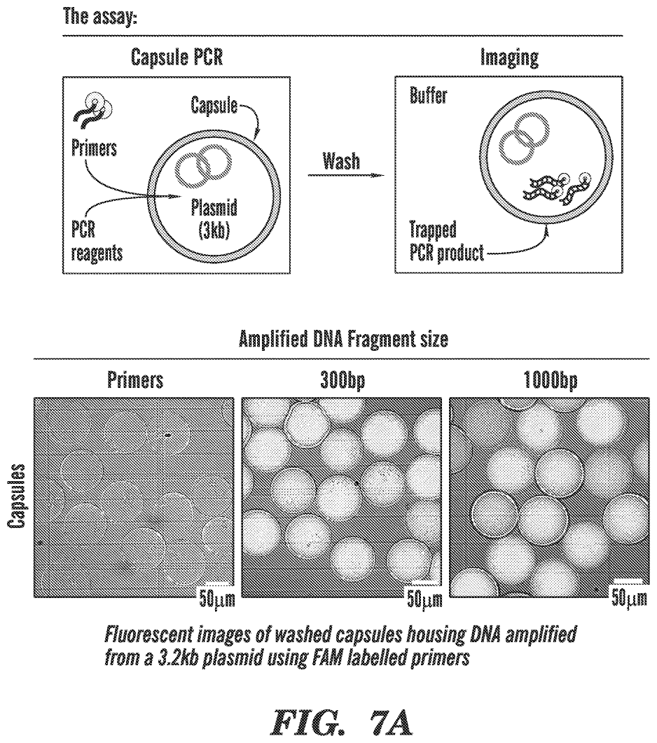

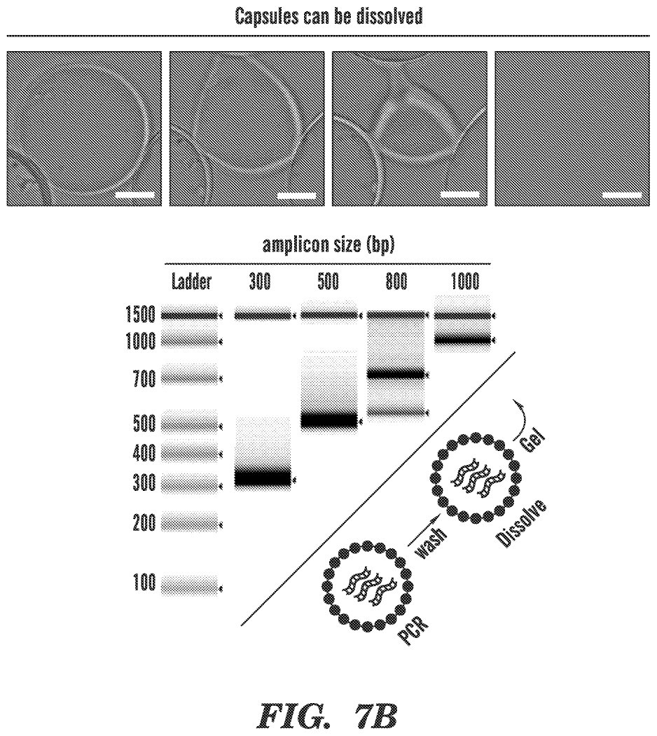

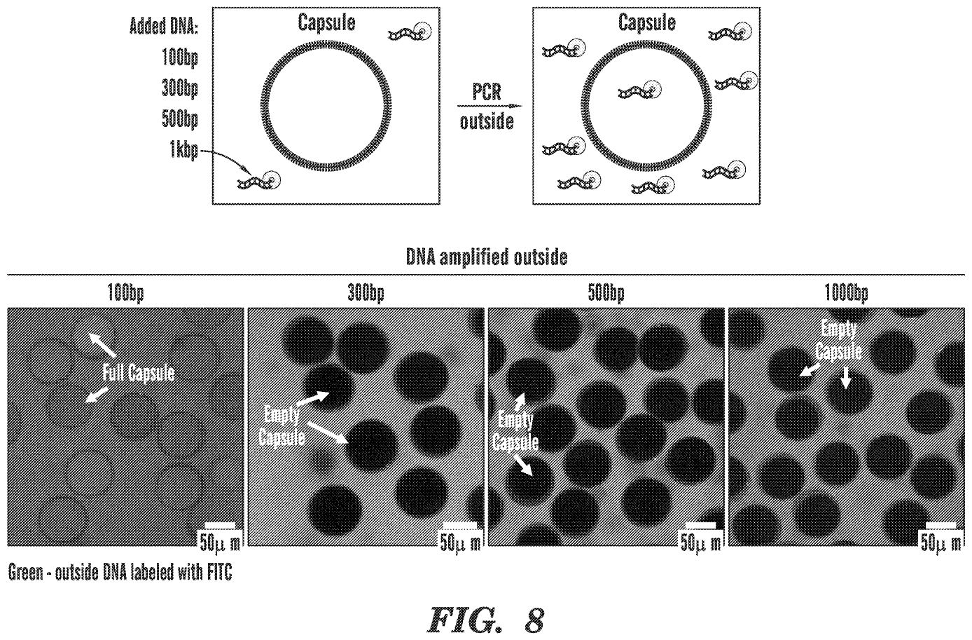

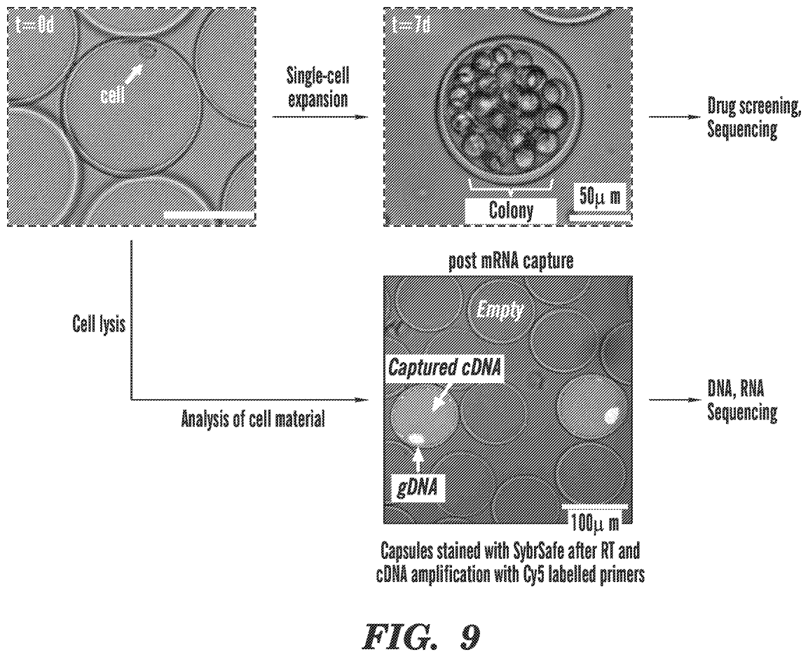

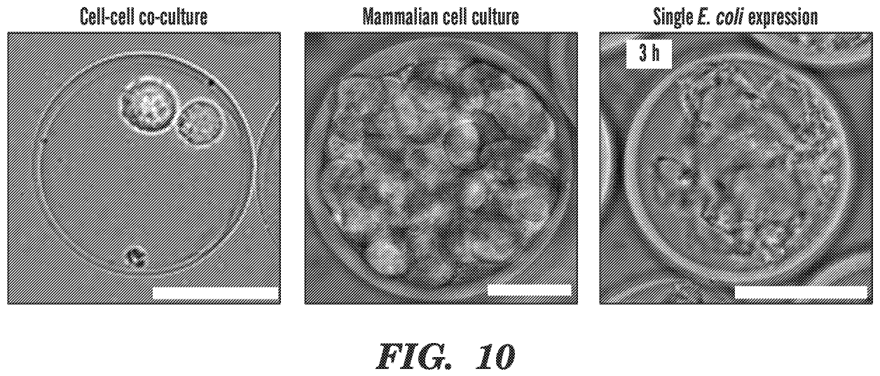

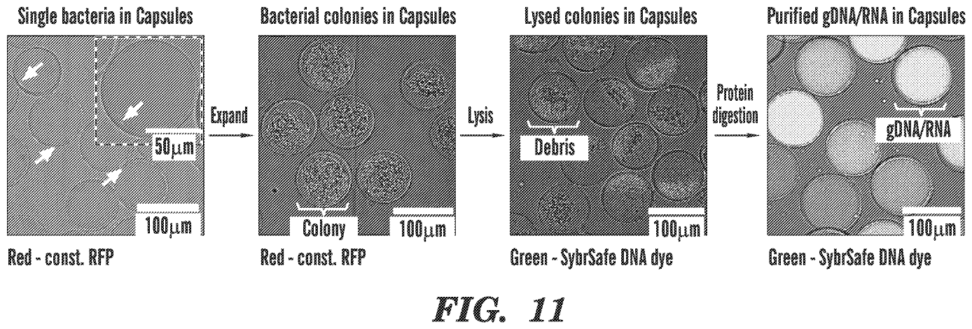

Hydrogel capsules synthesized by flowing 8% F127DA in DPBS and varied % of Dextran (MW=500 kDa) stably retain >300 nt nucleic acids while allowing proteins and short oligonucleotides to diffuse rapidly [FIG. 5A]. This enables enzymatic reactions to be performed on nucleic acids retained inside. An example of such experiment is PCR amplification of encapsulated DNA [FIG. 7A].

1 μL of plasmid DNA (1 ng/μL) was resuspended in the core solution composed out of 11% (w/w) Dextran (MW=500 kDa) in DPBS in 300 μL. Capsules were synthesized by encapsulating core mix with the shell mix (8% (w/w) F127DA in DPBS) at 1:1 ratio into ~80 μm droplets for 20 mins. After encapsulation, the emulsion (~200 μL) was stored on ice for 5 mins, followed by 30 °C for 3 mins. Photoinitiator was added on to the emulsion, followed by 5 s shaking and incubation at 30 °C for 3 mins. Capsules were polymerized by exposing the emulsion to 405 nm light for 5 mins.

Capsules were released from the emulsion by shaking with 20% Perfluoro-octanol, removing oil and washing once with Hexane. After the extraction, capsules were washed several times with 10 mM Tris+0.1% (v/v) Igepal CA-630.

### PCR Amplification of Encapsulated DNA

DNA amplicons of different sizes were amplified from the plasmid template inside the hydrogel capsules by adding the capsules to a PCR mix housing a universal FITC labeled forward primer and different RV primers. PCR conditions are summarized in Table 1. And carrying out a cycling program shown in Table 2.

TABLE 1

| Component | 20 μl Reaction | Final Concentration | |
|---|---|---|---|
| Nuclease-free water | 3.4 | | |
| 5X Phusion Buffer | 4 μL | 1X | |
| 10 mM dNTPs | 0.4 μL | 200 | μM |
| 10 μM FITC-FW primer | 1 μL | 0.5 | μM |
| 10 μM RV primer | 1 μL | 0.5 | μM |
| Capsules | 10 μL | | |
| Phusion DNA Polymerase | 0.2 μL | 0.4 | units |

| | |
|---|---|
| Final | 20 μL |

TABLE 2

| Step | Temperature | Time | |
|---|---|---|---|
| Initial Denaturation | 98 °C | 30 | s |
| 30 Cycles | 98 °C | 5 | s |
| | 70 °C | 15 | s |
| | 72 °C | 30 | s |
| Final Extension | 72 °C | 5 | min |

| | |
|---|---|
| Hold | 4 °C |

Hydrogel capsules were washed 5 times with 10 mM Tris+0.1% (v/v) Igepal CA-630 and were imaged using fluorescence microscope to verify the amplification and retention of dsDNA amplicons (visualized by incorporated FITC fluorescence) [FIG. 7A].

We also showed that empty capsules do not uptake DNA from the surroundings even at elevated temperatures by performing PCR of varied size DNA amplicons in the solution outside the capsules [FIG. 8].

### Recovery of in Capsule Amplified DNA

Hydrogel capsules housing DNA amplicons were dissolved by incubating at 37 °C in the presence of 1M NaOH and 1% Triton X-100. After solubilization, the solution was neutralized with HCl and 2× volume of AMPure beads were used to purify the DNA. DNA was analyzed using Bioanalyzer microcapillary electrophoresis [FIG. 7B].

### Additional Reactions in Capsules

The above examples can be extended to other reactions in which a substrate is retained in capsules while buffers, reaction co-factors and enzymes are introduced in one or more steps into capsules, and then removed by washing capsules after completion. In one example, RNA in capsules may be subject to reverse transcription. In another example, DNA in capsules may be subject to ligation. In a third example, chromatin in capsules may be subject to transposase-mediated fragmentation and ligation. In a fourth example, protein polymers may be labeled with antibodies.

### Cell Analysis in Hydrogel Capsules

Hydrogel capsules act as chamber that both permit growth of encapsulated cells and analysis of their molecular composition [FIG. 9]. For example, we showed that individual mammalian (such as L1210 [FIG. 9] and K562 [FIG. 10] cells) or bacterial (E. coli [FIGS. 10 and 11]) can be encapsulated into the capsules by resuspending them inside the core solution prior to droplet encapsulation and polymerization. During droplet production cells flow into the droplets and reside inside the liquid core after capsule polymerization. Cell housing capsules can be transferred to growth media for the subsequent in-capsule expansion. This enables a high-throughput interrogation of clonally expanded populations or co-cultured systems for perturbation, drug discovery and similar assays.

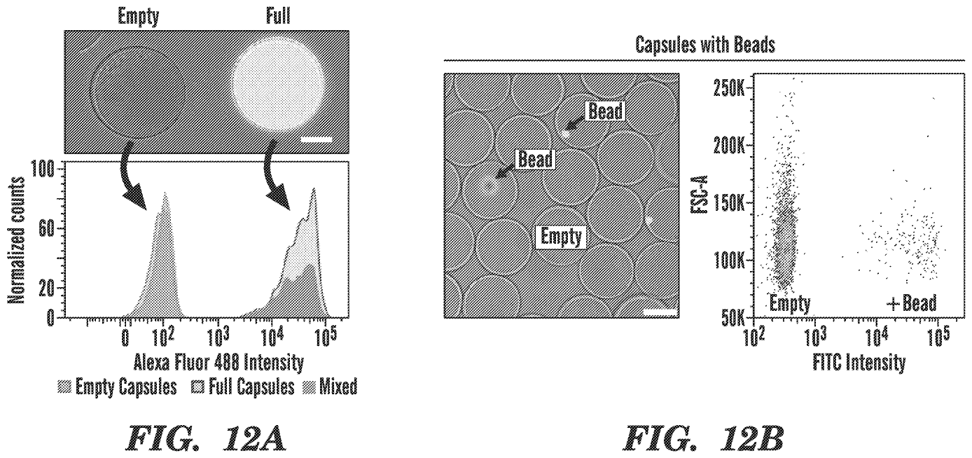

Specifically, we have shown that Pluronic capsules are suitable for cell culture [FIGS. 9, 10, and 31], flow cytometry [FIGS. 12A and 12B], imaging, and multi-step enzymatic reactions [FIGS. 11, 14A-14C, 18A-18C, and 19A-19B].

### Expansion of Cell in Capsules

For example, bacterial cells contain negligible amount of RNA and DNA per individual cells, making analysis of individual cells difficult. Analysis of expanded individual cells is limited by the infrastructure of colony handling, and at most enable processing up to hundreds of colonies per micro-well plate. In one example, we showed that each hydrogel capsules can act as bioreactor for clonal expansion of bacteria [FIG. 11]. Briefly, exponential growth phase E. coli were resuspended in 11% (w/w) Dextran (MW 500 kDa) in DPBS and were flown into ~80 μm droplets together with 8% (w/w) F127DA, 1% (w/w) PEG2000DA in DPBS at 1:1 ratio for 20 mins. After encapsulation, the emulsion (~200 μL) was stored on ice for 5 mins, followed by 30 °C for 3 mins. BAPO Photoinitiator was added on to the emulsion, followed by 5 s shaking and incubation at 30 °C for 3 mins. Capsules were polymerized by exposing the emulsion to 405 nm light for 5 mins. Capsules were released from the emulsion by shaking with 20% Perfluoro-octanol, removing oil and washing once with Hexane. After the extraction, capsules were washed several times with LB growth media with 0.1% (v/v) Pluronic L31. Single bacterial cells were expanded into colonies by incubating in 37 °C for several hours with shaking. The expanded colonies can then be used for single colony gDNA or RNA sequencing by lysing the cells with lysis buffer (such as lysozyme and SDS) followed by the removal of cellular debris using Proteinase K digestion. This results in capsule housing RNA and DNA of expanded colonies that are suitable for downstream analysis (genome, total RNA or transcriptome sequencing).

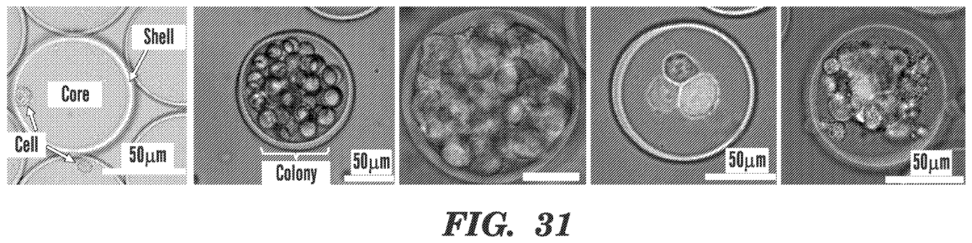

In another example, we showed that mammalian cell can be cultured in hydrogel capsules. L1210 cells were by resuspended in 11% (w/w) Dextran (MW 500 kDa) in DPBS and were flown into ~80 μm droplets together with 8% (w/w) F127DA, 1% (w/w) PEG2000DA in DPBS at 1:1 ratio for 20 mins. After encapsulation, the emulsion (~200 μL) was stored on ice for 5 mins, followed by 30 °C for 3 mins. BAPO Photoinitiator was added on to the emulsion, followed by 5 s shaking and incubation at 30 °C for 3 mins. Capsules were polymerized by exposing the emulsion to 405 nm light for 5 mins. Capsules were released from the emulsion by shaking with 20% Perfluoro-octanol, removing oil and washing once with Hexane. After the extraction, capsules were washed several times with DPBS with 0.1% (v/v) Pluronic L31 and then were placed in media (DMEM+10% FBS, 1×Pen/Strep) and transferred to a 5% CO2, 37 °C incubator. First to planes of FIG. 31 show microscopy images of capsules prior to growth and colonies formed after ~7 days.

### Single-Cell Analysis in Capsules

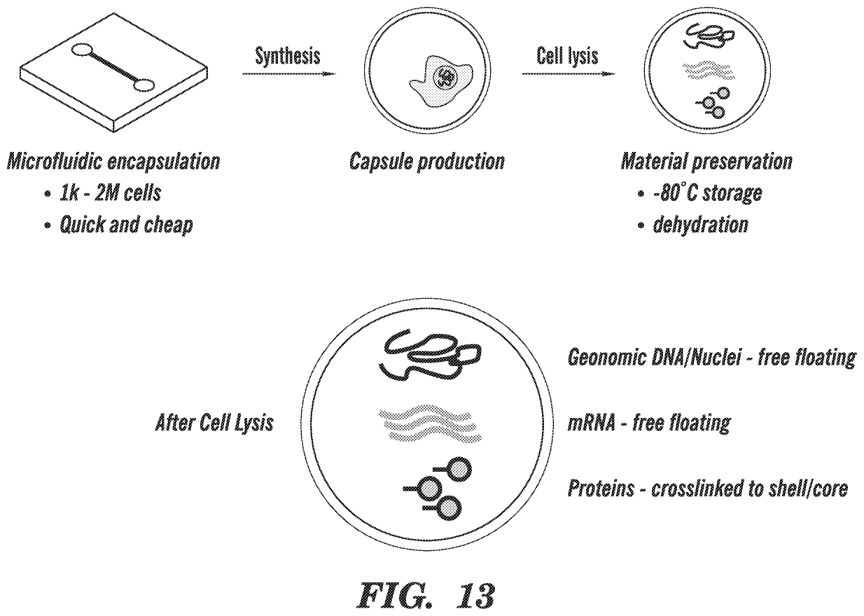

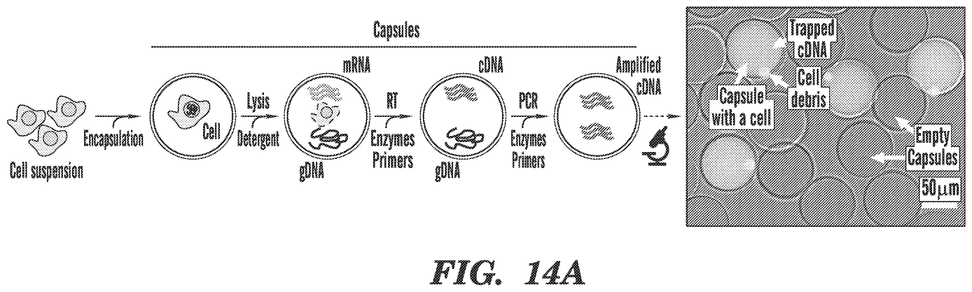

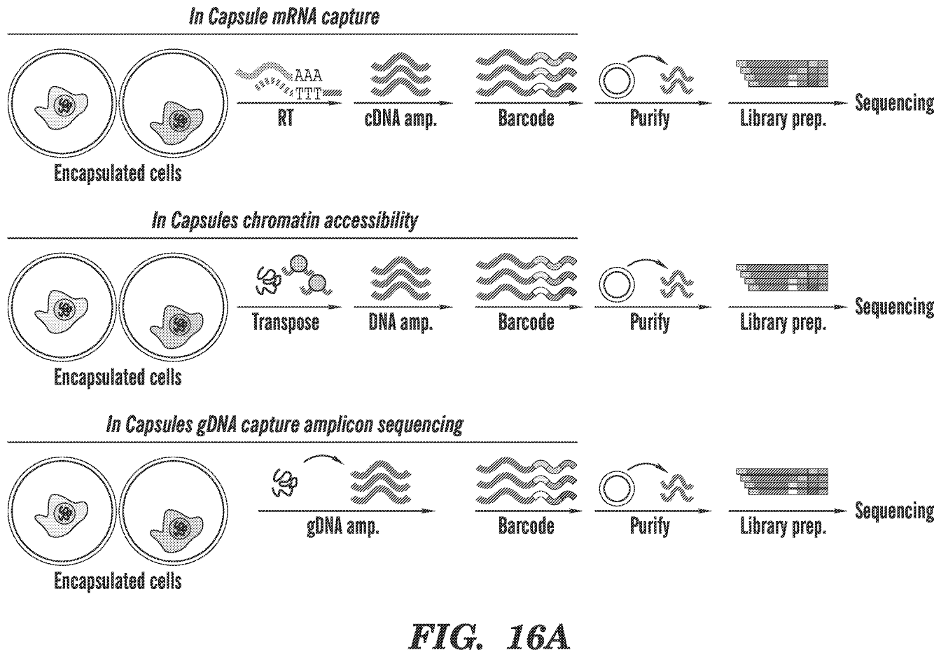

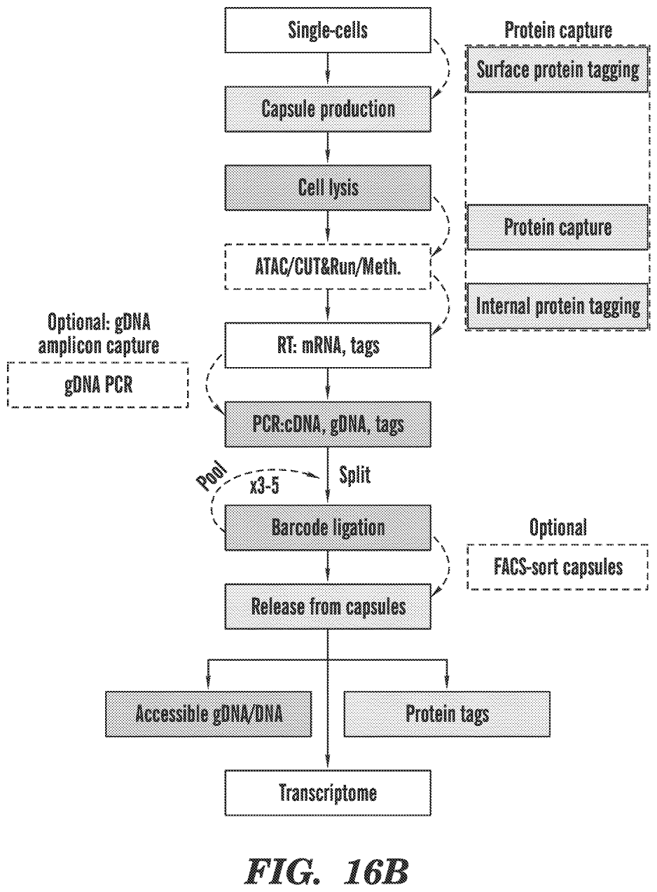

Single-cell sequencing protocols use barcoded DNA as a proxy to readout and assign the quantities of specific analytes to individual cells. Thus, the efficiency of molecular reactions used to generate this DNA defines the assays sensitivity. This is specifically important in split-pool based approaches that use multiple rounds of combinatorial DNA addition to construct a unique barcode. Failure to append at least one tag results in loss of data. We argue that hydrogel capsules act as micro-reactors for high-quality processing of single-cell derived material (such as mRNA, native chromatin DNA, gDNA modifications) and enable the use of split-pool barcode addition after the amplification step, thus minimizing the molecule loss inherent to such barcoding methods. Hydrogel capsules contain analytes from single-cells and are amenable majority of assays [FIGS. 13 and 16A-16B].

### Capsule Based Single-Cell RNA-Seq

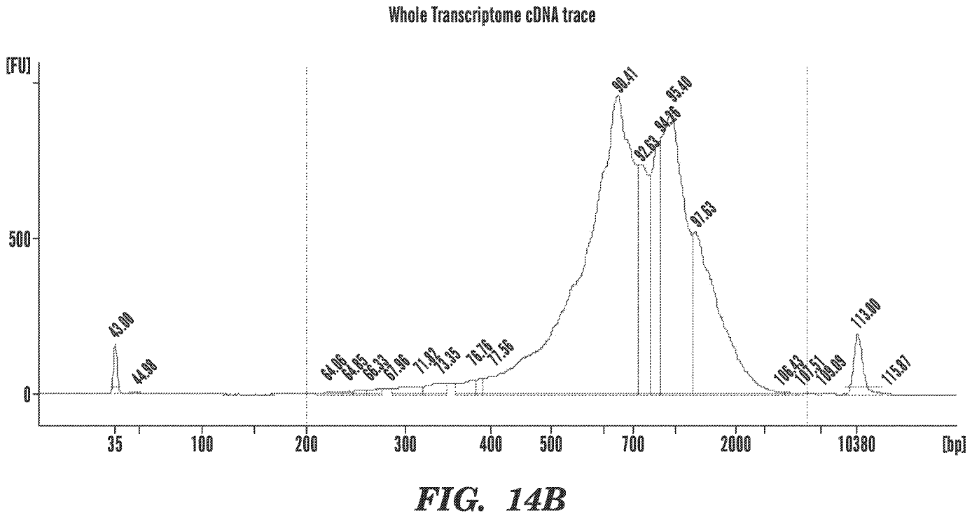

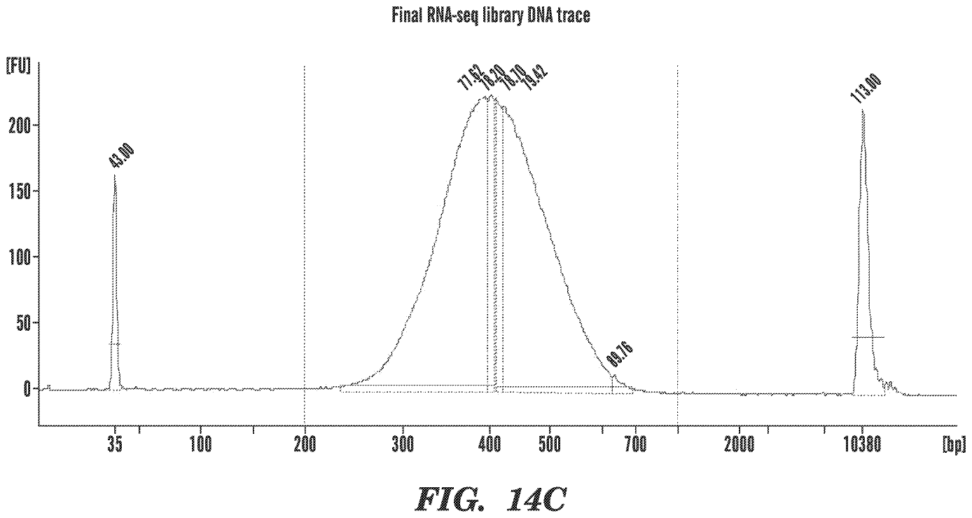

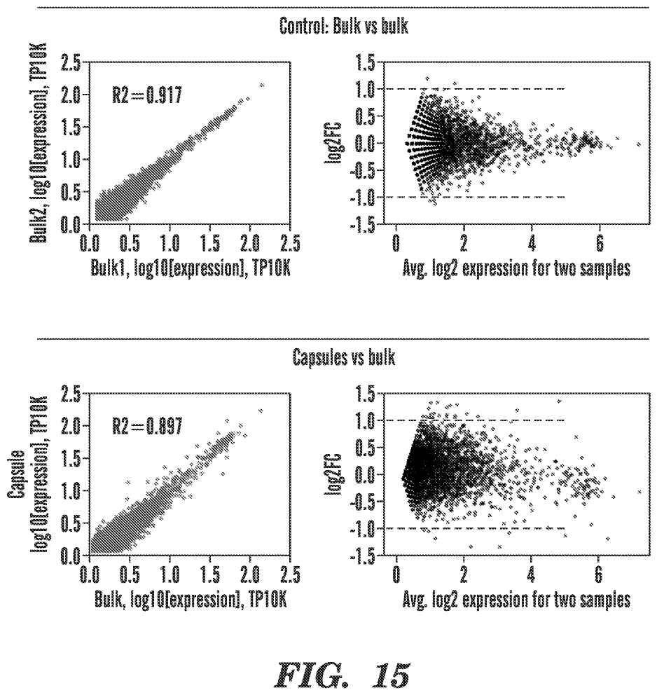

For example, a suspension of single cells can be encapsulated into hydrogel microcapsules such that the average occupancy of capsules is <0.1 cells/per capsule, leading to most cells being encapsulated in isolation of other cells. If cells enter capsules randomly, the distribution of cells per capsule will follow a Poisson distribution, such that when the average number of cells per capsule λ<<1 (e.g. λ=0.1 cells/capsule), then the fraction of capsules containing exactly 1 cell/capsule out of capsules containing at least 1 cell per capsule is approximately (1−λ/2) (e.g. 0.95 or 95% for λ=0.1). These capsules can then be mixed with a buffer containing cell lysis reagents including but not limited to surfactants, proteases, chaotropic salts, followed by a duplication of mRNA released from the lysed cells but retained in capsules to generate complementary DNA (cDNA), by first optionally washing the capsules to remove lysis reagents, followed by mixing the capsules with enzymes, buffer and cofactors required for a reverse-transcription reaction. The resulting cDNA can then be amplified [FIG. 14A]. In one such case, L1210 cells were resuspended in DPBS with 11% (w/w) Dextran (MW 500 kDa) in DPBS and were flown into ~80 μm droplets together with 8% (w/w) F127DA, 1% (w/w) PEG2000DA in DPBS at 1:1 ratio for 20 mins. After encapsulation, the emulsion (~200 μL) was stored on ice for 5 mins, followed by 30 °C for 3 mins. BAPO photoinitiator was added on to the emulsion, followed by 5 s shaking and incubation at 30 °C for 3 mins. Capsules were polymerized by exposing the emulsion to 405 nm light for 5 mins. Capsules were released from the emulsion by shaking with 20% Perfluoro-octanol, removing oil and washing once with Hexane. After the extraction, capsules were washed several times with 7.5 pH 10 mM Tris-HCl, 50 mM EDTA with 0.1% (v/v) Igepal CA-630. Cells were lysed on ice for 10 min in 10 mM Tris and 50 mM EDTA, 0.2% Triton X100, 0.3% Igepal Ca-630, 0.5 U/μL RNAse inhibitor. mRNA was captured using in-capsule reverse transcription (1× Maxima H-buffer, 0.5 mM dNTPs, 3 μM poly-T30VN primer, 0.3% Igepal CA-630, 20 μM Template switching oligo, 1 U/μL Ribolock RNAse inhibitor, 10 U/μL Maxima H-RTase), and cDNA amplification (NEBNext® Ultra™ II Q5® Master Mix). Single-cell derived whole-transcriptome cDNA was then purified [FIG. 14B] and used for next-generation sequencing library preparation (NEBNext® Ultra™ II DNA Library Prep Kit for Illumina®) [FIG. 14C]. Gene-expression analysis between cells processed in solution and in capsules show no significant deviation and establish that hydrogel capsules are suitable for multi-step single-cell processing without transcript loss [FIG. 15].

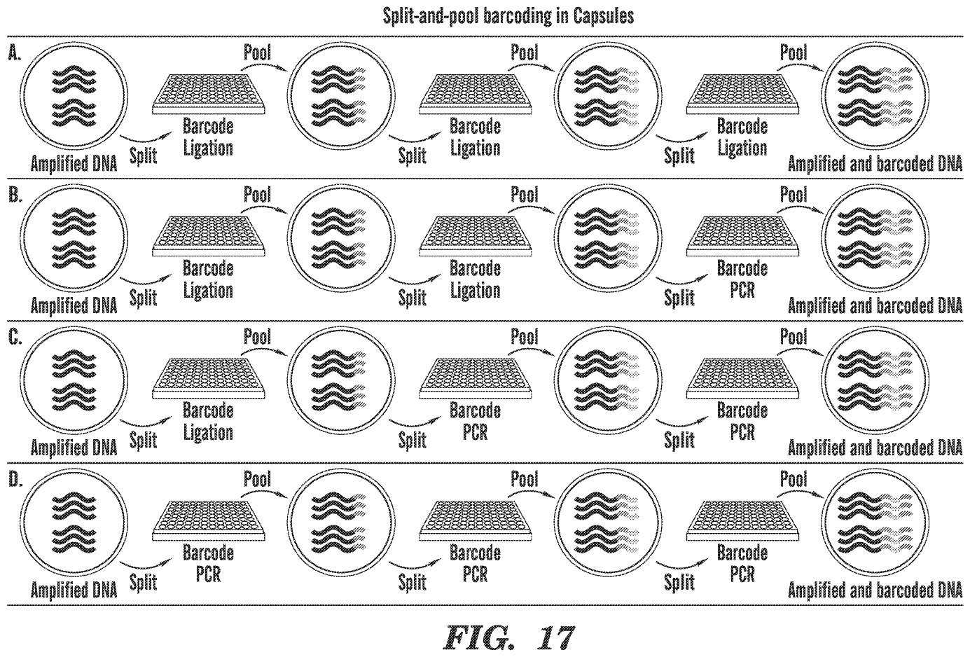

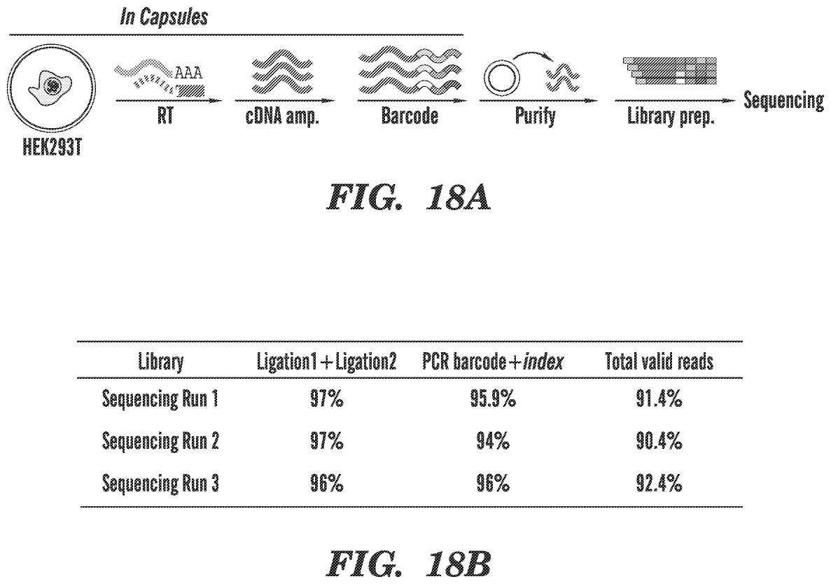

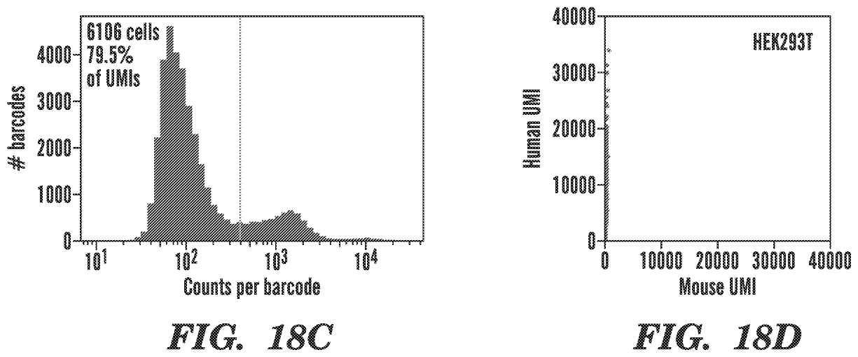

After successfully proving the ability to process cells in capsules we have implemented combinatorial DNA barcoding procedures [FIG. 17]. A capsule unique DNA barcode is constructed on the encapsulated DNA by several rounds of DNA sequence additions. As a result, molecules arising from a single-capsules (and thus from a single-cell) can be identified after sequencing, providing single-cell resolution. In most cases, barcodes are added on to the amplified DNA and avoids molecule loss due to inherent reaction efficiencies. In one such example, barcodes are added on to the amplified cDNA derived from individually encapsulated HEK293T cells in 2-ligation and 1-PCR round [FIG. 18A]. The method results in efficient barcoding with accurate reads structure [FIG. 18B] that enables the captured transcripts to be assigned to individual cells [FIGS. 18C and 18D].

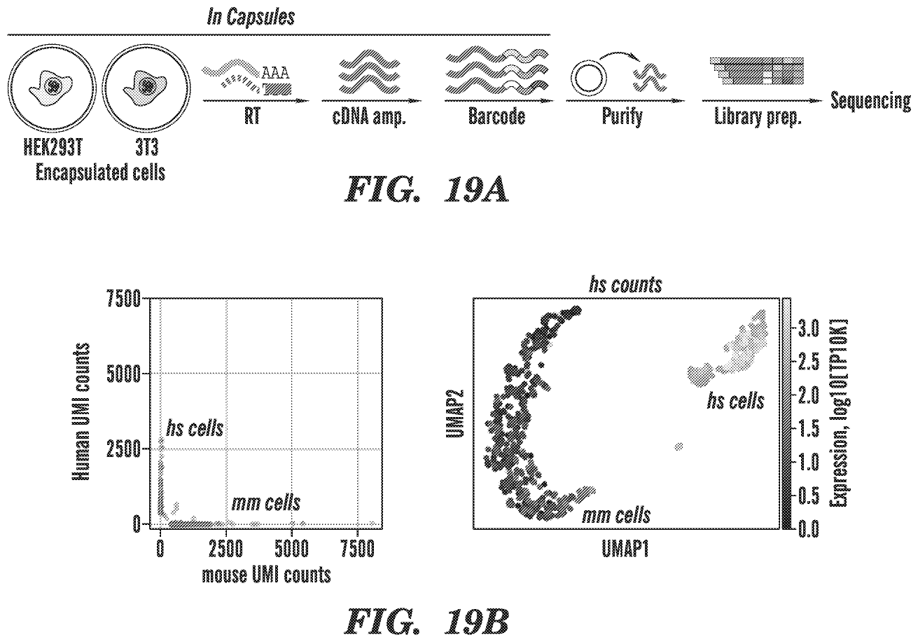

An important consideration for single-cell processing methods is the ability to isolate single-cell derived material from other cells. That is, there should be no cross-contamination between individual cells during all the processing and handling steps. We established that hydrogel capsules successfully retain transcripts by carrying out a species mixing experiments [FIG. 19A]. Briefly, a mixture of human and mouse cells were resuspended in DPBS with 11% (w/w) Dextran (MW 500 kDa) in DPBS and were flown into ~80 μm droplets together with 8% (w/w) F127DA, 1% (w/w) PEG2000DA in DPBS at 1:1 ratio for 20 mins. After encapsulation, the emulsion (~200 μL) was stored on ice for 5 mins, followed by 30 °C for 3 mins. BAPO photoinitiator was added on to the emulsion, followed by 5 s shaking and incubation at 30 °C for 3 mins. Capsules were polymerized by exposing the emulsion to 405 nm light for 5 mins. Capsules were released from the emulsion by shaking with 20% Perfluoro-octanol, removing oil and washing once with Hexane. After the extraction, capsules were washed several times with 7.5 pH 10 mM Tris-HCl, 50 mM EDTA with 0.1% (v/v) Igepal CA-630. Cells were lysed on ice for 10 min in 10 mM Tris and 50 mM EDTA, 0.2% Triton X100, 0.3% Igepal CA-630, 0.5 U/μL RNAse inhibitor. mRNA was captured using in-capsule reverse transcription (1× Maxima H-buffer, 0.5 mM dNTPs, 3 μM poly-T30VN primer, 0.3% Igepal CA-630, 20 μM Template switching oligo, 1 U/μL Ribolock RNAse inhibitor, 10 U/μL Maxima H-RTase), cDNA amplification (NEBNext® Q5UR Master Mix), followed by 3-rounds of barcoding, DNA recovery and single-cell RNA-seq library construction (NEBNext® Ultra™ II DNA Library Prep Kit for Illumina®). The results show that individual capsules only contain transcripts (counter as reads with unique molecular identifier (UMI)) arising from human or mouse cells (except for a few doublet containing capsules) [FIG. 19B] and result in two clearly separating human and mouse populations. This data establishes hydrogel capsules as a platform for high-throughput single-cell gene expression analysis.

### Capsule Based Chromatin Accessibility

Profiling the chromatin accessibly of single cells is an alternative approach to scRNA-seq by which to define cell types and cell states. Linking it to single-cell transcriptomic information enables association of enhancer elements with target genes. The ability to measure both types of information from the same cell enables direct identification of co-varying genomic loci for differentially expressed transcripts. While there are a handful of recently developed methods that enable this type of joint measurement in large numbers of single cells, these protocols rely on either cell fixation or cell-type specific membrane permeabilization, shown to interfere with methods sensitivity. For example, isolated nuclei based scATAC-seq protocols exhibit 2-5-fold greater unique DNA fragment counts in comparison to methods using permeabilized or cross-linked cells. However, the use of nuclei results in substantial loss of mature mRNAs. Similarly, permeabilization-based approaches underperform when compared to approaches done with fresh cells^{14,15,24,38}. Capsules present a solution to these technical challenges, whereby cell lysis and subsequent DNA-directed transposon insertion can be performed without loss of cytoplasmic transcripts, such that libraries reporting on both the genome and transcriptome can be reliably generated for many individual cells.

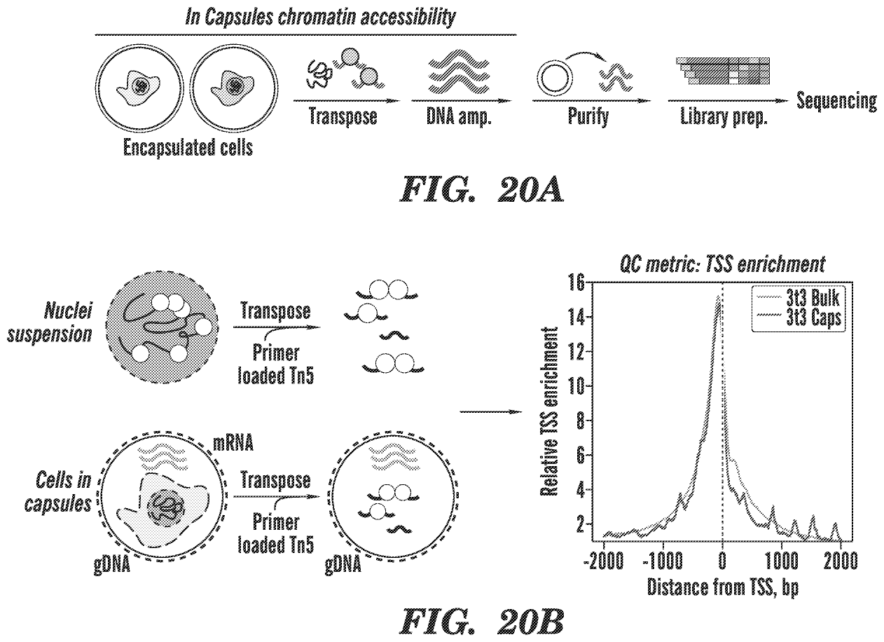

The ability to carry out native chromatin accessibility assay in capsules was showcased using NIH 313 cells [FIGS. 20A and 20B]. Briefly, cell were encapsulated into hydrogel capsules as described above. Cells were lysed using cold lysis buffer (10 mM 7.4 pH Tris-HCl, 10 mM NaCl, 3 mM MgCl₂, 0.1% Igepal CA-630, 0.1% Tween-20, 0.01% Digitonin, 1% BSA) for 4 mins. Next, transposition reaction mix (from Nextera DNA Library Prep Kit) was added and the sample was incubated incubate at 37 °C for 30 minutes. DNA was amplified in capsules (NEBNext High-Fidelity 2×PCR Master Mix) and DNA was isolated using Qiagen MinElute Reaction Cleanup Kit, followed by final library PCR (NEBNext High-Fidelity 2×PCR Master Mix) and sequencing [FIG. 20B]. Data show that resulting reads show similar transcription start site (TSS) enrichment scores between nuclei processed in solution or cells in capsules, highlighting the usability of hydrogel capsules to carry out chromatin accessibility assays.

### Preservation of Material

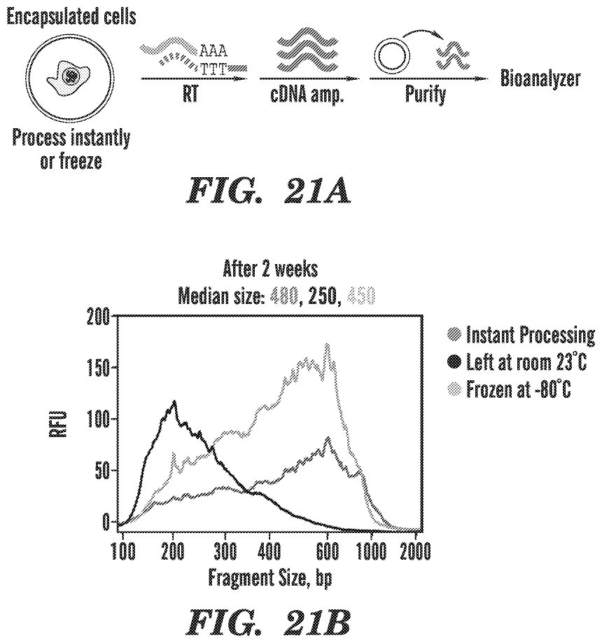

Hydrogel capsules stably retain longer molecules such as RNA and DNA and are robust to harsh chemical lysis. This hints that nucleic acids can be purified from cells and stably retained in hydrogel capsules for long-term storage. In one such example, we showcase the ability to preserve RNA in capsules after cell lysis [FIG. 21]. Briefly, NIH 3t3 cells were encapsulated into ~90 μm capsules composed out of 11% (w/w) Dextran (MW 500 kDa) in DPBS and 8% (w/w) F127DA, 1% (w/w) PEG2000DA in DPBS mixed at 1:1 ratio [FIG. 21A]. The capsules where washed serval times with 7.5 pH 10 mM Tris-HCl, 50 mM EDTA with 0.1% (v/v) Igepal CA-630 followed by lysis with 0.3% (v/v) SDS on ice for 10 min and more washes. Resulting capsules were split into three tubes. One tube was instantly processed using in-capsule reverse transcription (1× Maxima H-buffer, 0.5 mM dNTPs, 3 μM poly-T30VN primer, 0.3% Igepal CA-630, 20 μM Template switching oligo, 1 U/μL Ribolock RNAse inhibitor, 10 U/μL Maxima H-RTase), and cDNA amplification (NEBNext® Ultra™ II Q5® Master Mix). The other two tubes were either left at room temperature or frozen down at 80 °C. After two weeks, the two tubes were washed and processed in the same way as the instant tube. Whole-transcriptome cDNA was purified, and fragment size was analyzed [FIG. 21B]. Data shows that capsules left at room temp have degraded RNA (cDNA lengths shifted to short lengths), while the instantly processed or frozen sample contain long-length cDNA molecules, corresponding to undegraded transcripts.

### Capsule Immobilization for Sequential Analysis

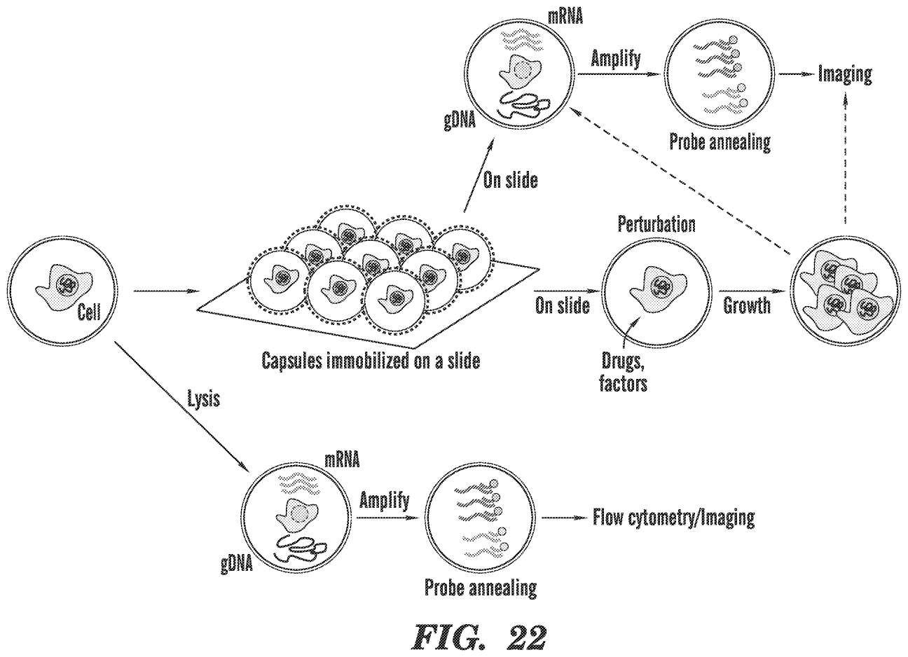

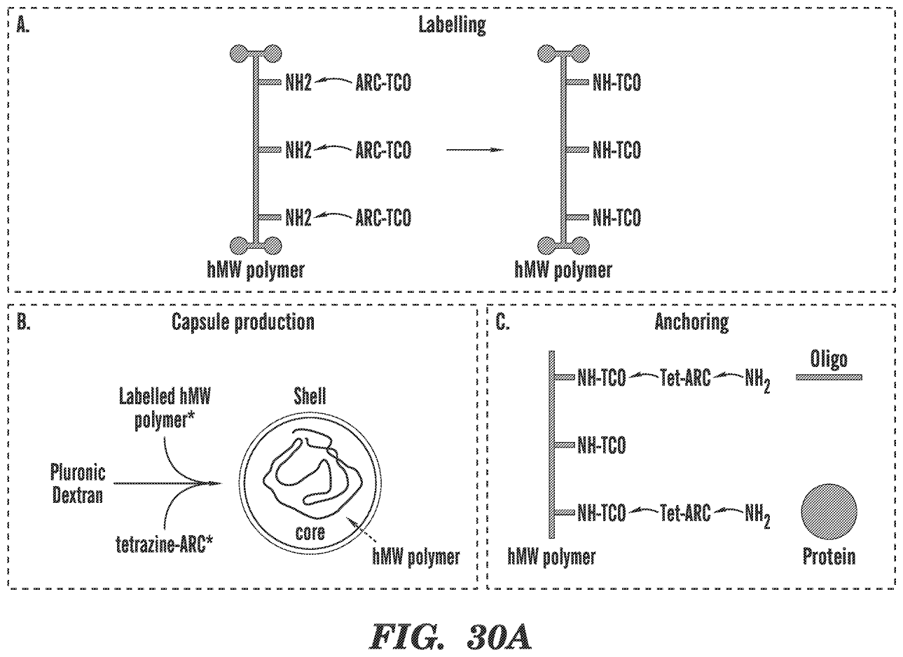

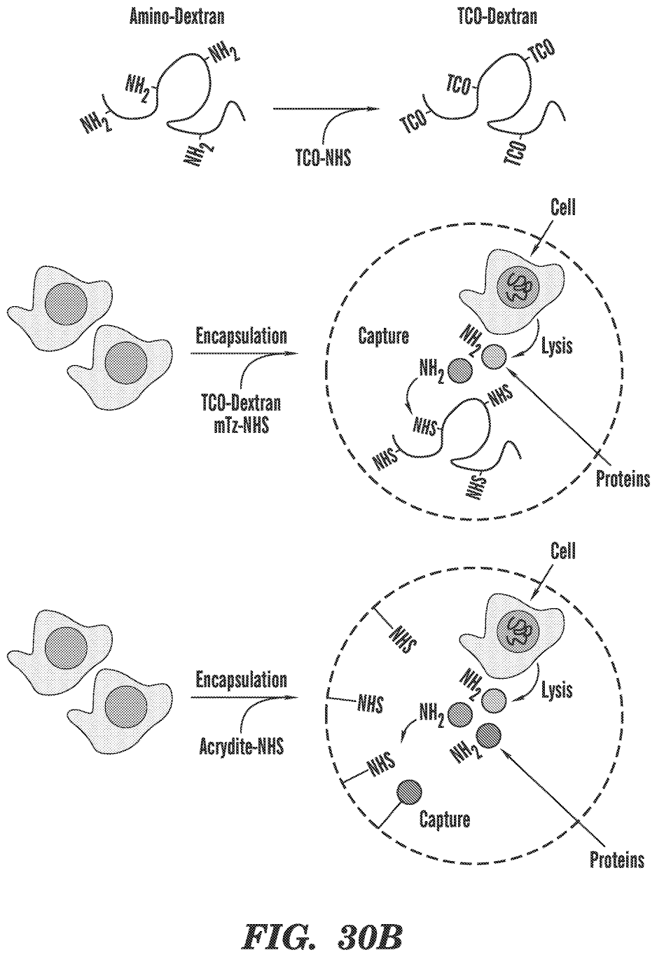

In addition to sequencing, hydrogel capsules provide way to rapidly probe gene expression in a sequencing free manner, as well as to track cell dynamics in multiple isolated capsules in parallel. For example, a number of probes can be directly annealed to mRNA or amplified cDNA derived from individually encapsulated cells. Flow cytometry can be used to measure the intensity and type of fluorescence to quickly characterize the gene expression of a set number of genes. Moreover, hydrogel capsules can be supplemented polymers which can be chemically modified [FIGS. 30A and 30B]. In one example, amino-PEG2000-acrylate was supplemented into hydrogel shell to facilitate binding of N-hydroxysuccinimide (NHS) ester modified methyl-tetrazine (mTz) click chemistry reagents. Resulting capsules are coated with mTz and can react with trans-cyclooctene (TCO) via an inverse-demand Diels-Alder cycloaddition reaction. In one such case, capsules were immobilized on a glass surface coated with TCO. This enables quick micro-reactor array formation on a glass surface for high-throughput microscopy of cultured cells followed by gene expression probing [FIG. 22].

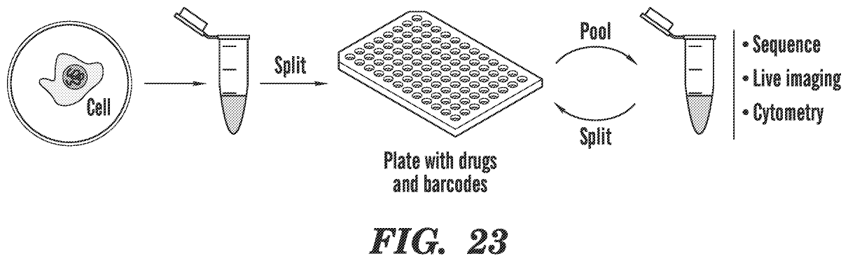

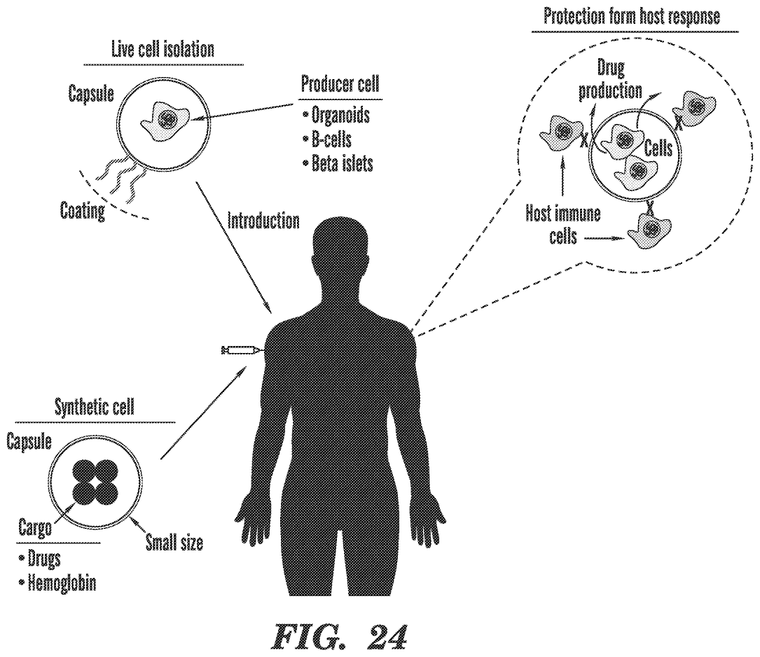

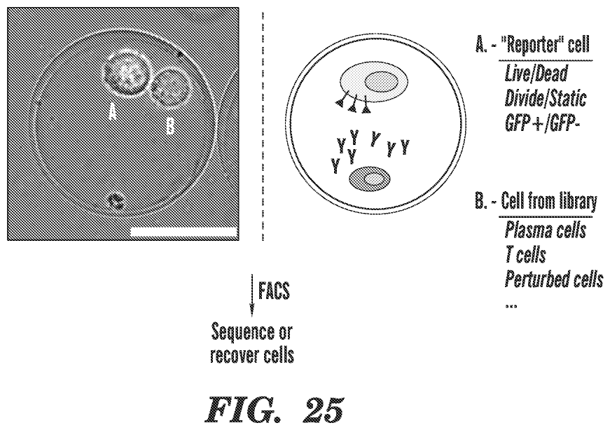

With the capability to culture cells inside micro-compartments and then read out the contents using microscopy, sequencing or flow cytometry, hydrogel capsules constitute a platform for high-throughput screening by sequential analysis. In one example, live cells can be encapsulated into hydrogel capsules and then sequentially exposed to different types of perturbations [FIG. 23] with each perturbation appending a DNA barcode to the capsule. A series of perturbation steps can be employed to combinatorically expose cells to different compounds. Cytometry or sequencing methods can then be used to read out the state of the exposed cells and link them to the perturbations received. An example of such assay can be used to identify the compounds needed to differentiate a cell to a certain outcome for research or clinical use. In another case, two types of cells can be placed in the same capsule and their interaction can be used as a readout to screen for functional cellular phenotype (etc. CAR-T and cancer cells or functional antibodies ability to modulate the phenotype of a reporter cell) [FIG. 25]. In such case, one or a group of cells act as a ‘reporter’ and produces a signal (growth, fluorescence or etc.) to report on the activity (killing, antibody induced activation/inhibition) of another cell, that is an isolated primary cell or a cell from a library candidate engineered cells.

### Capsule Immobilization of Small Proteins for Cytometry

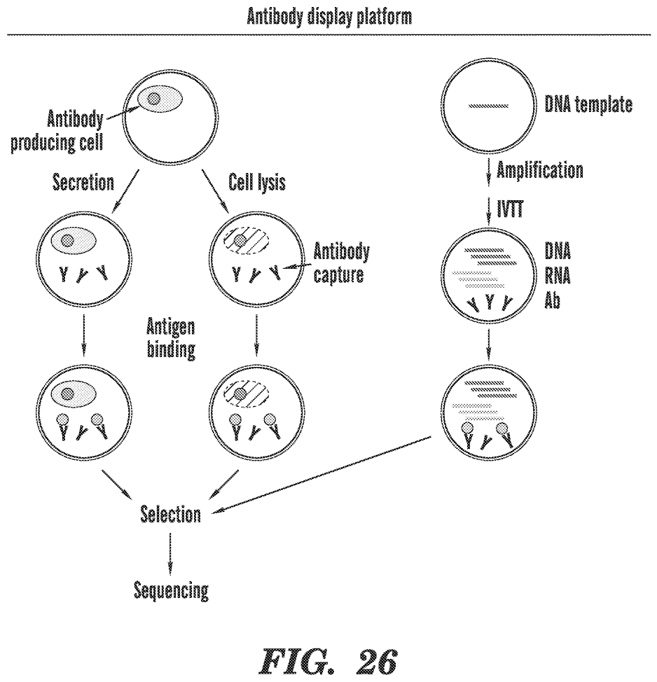

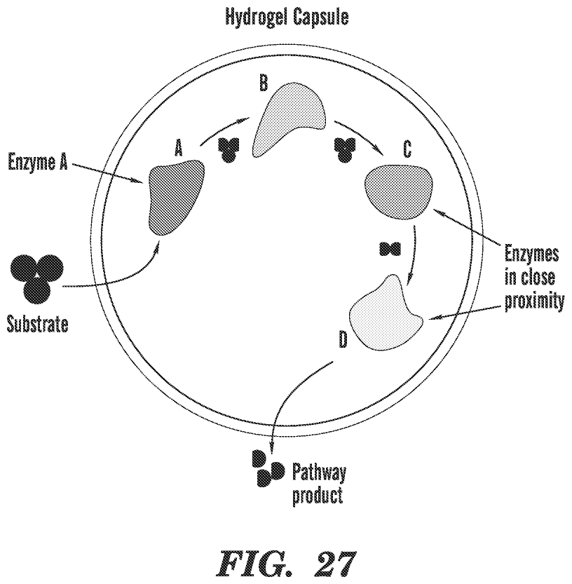

Cell analytes that normally diffuse out of the capsule may be immobilized inside the capsules core via the linkage to high-molecular weight dextran. For instance, anti-immunoglobulin antibodies can be introduced to the core to act as anchor for antibody screening [FIG. 26]. In one of such cases, capsules are first produced housing immobilized anti-immunoglobulin antibodies and individual cells secreting different variants of immunoglobulins. The secreted antibodies will be captured by the anchored ones and will be displayed inside the capsule. Addition of labeled target analyte enables the selection of capsules housing antibodies with high affinity.

### Cell Immune Isolation in Pluronic Micro-Capsules

It has been shown that Pluronic® are biocompatible and do not elicit an immune response. In one possible application, hydrogel capsules are used as compartments to isolate therapeutic cell or compounds from the host tissue. In such case, therapeutic cells (for example, Beta islets) or compounds (for example, slow releasing drugs) are encapsulated into capsules during their production. Resulting micro-capsules are introduced into a host (a human patient or an animal) via injection or tissue engraftment and provide physical barrier from the surrounding tissue and immune system.

### Enzymatic Cascades in Pluronic Micro-Capsules

In another instance, hydrogel capsules are used as micro-reactors to house enzymes in proximity for complex reaction cascades [FIG. 27]. In such case, enzymes of interest are linked to amino dextran (for example, via methyl-tetrazine and trans-cyclooctene assisted click chemistry reaction [FIGS. 30A and 30B]). Enzyme coupled dextran is supplemented into core solution during capsule production. Resulting capsules house immobilized enzymes and can be placed as units into solutions containing analytes to carry out reactions. For example, such capsules can be placed into bioreactors where analytes of interest are produced by the growing biomass. Hydrogel capsules act as physical barrier that separates cells from the enzymes, removing toxicity to cells or reaction inhibition. Moreover, capsules can be recovered, washed and reused.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

What is claimed is:

1. A capsule comprising:
(a) a liquid core; and
(b) a shell surrounding the liquid core, wherein the shell comprises a cross-linked poly(ethylene glycol)-poly(propylene oxide)-poly(ethylene glycol) diacrylate triblock copolymer.

2. The capsule of claim 1, wherein the shell is semi-permeable.

3. The capsule of claim 1 or 2, wherein the core comprises a solute.

4. The capsule of claim 3, wherein the solute is a viscosity modifier.

5. The capsule of claim 3 or 4, wherein the solute is a sugar.

6. The capsule of any one of claims 3-5, wherein the solute is dextran.

7. The capsule of claim 6, wherein the dextran has a molecular weight in the range of about 3000 Daltons to about 2,000,000 Daltons.

8. The capsule of any one of claims 1-7, wherein the capsule is a microcapsule.

9. The capsule of any one of claims 1-8, wherein the capsule has a diameter of from about 1 μm to about 1000 μm (e.g., a diameter of 1 μm to about 500 μm, from about 1 μm to about 300 μm or from about 5 μm to about 250 μm).

10. The capsule of any one of claims 1-8, wherein the capsule has a diameter of from about 5 μm to about 200 μm.

11. The capsule of any one of claims 1-10, wherein the capsule is optically clear.

12. The capsule of any one of claims 1-11, further comprising a coating covering an outer surface of the shell, wherein the coating is a biocompatible material.

13. The capsule of claim 12, wherein the biocompatible material is a fluorinated compound, polyethylene glycol (PEG), poloxamer, gelatin, silanizing agent, collagen, fibrinogen, laminin, or bovine serum albumin (BSA).

14. The capsule of any one of claims 1-13, further comprising an agent.

15. The capsule of claim 14, wherein the agent is in the core.

16. The capsule of claim 14, wherein the agent is in the shell.

17. The capsule of any one of claims 14-16, wherein the agent is a cell, biomolecule, polymer, small organic or inorganic molecule, microorganism or organoid.

18. The capsule of any one of claims 14-16, wherein the agent is an amino acid, peptide, polypeptide, nucleotide, oligonucleotide, polynucleotide, saccharide, oligosaccharide, or polysaccharide.

19. The capsule of any one of claims 14-16, wherein the agent is an enzyme, an antibody, a primer nucleic acid, or a plasmid.

20. The capsule of any one of claims 14-16, wherein the agent is a cell.

21. The capsule of any one of claims 14-16, wherein the agent is a therapeutic agent or an imaging agent.

22. The capsule of any one of claims 1-21, further comprising a high molecular weight molecule in the core.

23. The capsule of claim 22, wherein the high molecular weight molecule comprises at least one functional group for forming a linkage with an agent.

24. The capsule of claim 22 or 23, wherein the high molecular weight molecule is a high molecular weight polymer.

25. The capsule of any one of claims 1-24, further comprising a cross-linking molecule in the shell.

26. The capsule of claim 25, wherein the cross-linking molecule comprises at least one functional group for forming a linkage with an agent.

27. A method for preparing a capsule, the method comprising:
(i) forming an emulsion comprising droplets of an aqueous phase solution disposed in a non-aqueous phase solution, wherein the droplets comprise a liquid core surrounded by a shell comprising an uncross-linked polymer blend, wherein the uncross-linked polymer blend comprises an uncross-linked poly(ethylene glycol)-poly(propylene glycol)-poly(ethylene glycol) diacrylate triblock copolymer and an uncross-linked poly(ethylene glycol) diacrylate homopolymer; and
(ii) cross-linking the polymer blend, and
wherein the capsule comprises: (a) a liquid core; and (b) a shell surrounding the liquid core, wherein the shell comprises a cross-linked polymer blend, wherein the cross-linked polymer blend comprises a cross-linked poly(ethylene glycol)-poly(propylene glycol)-poly(ethylene glycol) diacrylate triblock copolymer and cross-linked poly(ethylene glycol) diacrylate homopolymer.

28. The method of claim 27, wherein a ratio of homopolymer to triblock copolymer is from about 99:1 to about 1:99 (w/w or v/v). (e.g.,

29. The method of claim 27 or 28, wherein an amount of the homopolymer is higher relative to an amount of the triblock copolymer (w/w or v/v).

30. The method of any one of claims 27-29, wherein the step of forming the emulsion comprises mixing the aqueous phase solution with the uncross-linked polymer blend to form a first mixture and mixing the first mixture with a non-aqueous phase solution.

31. The method of any one of claims 27-30, wherein the step of forming the emulsion is in a microfluidic device.

32. The method of any one of claims 27-29, wherein the step of forming the emulsion comprises:
feeding the aqueous phase solution into a first microfluidic channel of a microfluidic device;
feeding the un-crosslinked polymer blend into a second microfluidic channel of the microfluidic device;
feeding the non-aqueous phase solution into a fourth microfluidic channel of the microfluidic device;
mixing the aqueous phase solution with the uncross-linked polymer blend at a first intersection between the first microfluidic channel and the second microfluidic channel to form a first mixture;
conveying the first mixture through a third microfluidic channel toward a second intersection between the third microfluidic channel and the fourth microfluidic channel;
mixing the first mixture with the non-aqueous phase solution at the second intersection to form the emulsion; and
collecting the emulsion from the microfluidic device.

33. The method of any one of claims 27-32, wherein the cross-linking comprises photo-cross-linking.

34. The method of any one of claims 27-33, wherein said cross-linking comprises exposing the emulsion to ultraviolet (UV) radiation or visible light radiation.

35. The method of any one of claims 27-34, wherein the emulsion comprises a photoinitiator.

36. The method of claim 35, further comprising adding the photoinitiator to the emulsion prior to the step of cross-linking.

37. The method of claim 35 or 36, wherein the non-aqueous phase solution comprises a photoinitator.

38. The method of any one of claims 35-37, wherein the photoinitiator is a hydrophobic radical polymerization reaction photoinitiator.

39. The method of any one of claims 27-32, wherein the cross-linking comprises chemical cross-linking.

40. The method of any one of claims 27-39, wherein the non-aqueous phase solution comprises an oil.

41. The method of claim 40, wherein the oil is a fluorinated oil.

42. The method of claim 41, wherein the fluorinated oil is a fluorine-substituted alkylsiloxane or a fluorocarbon or a hydrofluoroether.

43. The method of any one of claims 27-42, wherein the cross-linked shell is semi-permeable.

44. The method of any one of claims 27-43, wherein the liquid core comprises a solute.

45. The method of claim 44, wherein the solute is a viscosity modifier.

46. The method of claim 44, wherein the solute is a sugar.

47. The method of claim 44, wherein the solute is dextran.

48. The method of claim 47, wherein the dextran has a molecular weight in the range of 3000 Daltons to 2,000,000 Daltons.

49. The method of any one of claims 27-48, wherein the capsule is a microcapsule.

50. The method of any one of claims 27-49, wherein the capsule has a diameter of 1 μm to 1000 μm.

51. The method of any one of claims 27-49, wherein the capsule has a diameter of 5 μm to 200 μm.

52. The method of any one of claims 27-51, wherein the capsule is optically clear.

53. The method of any one of claims 27-52, further comprising coating an outer surface of the cross-linked shell with a biocompatible material.

54. The method of any one of claims 27-53, further comprising adding an agent to the capsule.

55. The method of claim 54, wherein the agent is in the liquid core.

56. The method of claim 54, wherein the agent is in the cross-linked shell.

57. The method of any one of claims 54-56, wherein the agent is a cell, biomolecule, polymer, small organic or inorganic molecule, microorganism or organoid.

58. The method of any one of claims 54-56, wherein the agent is an amino acid, peptide, polypeptide, nucleotide, oligonucleotide, polynucleotide, saccharide, oligosaccharide, or polysaccharide.

59. The method of any one of claims 54-56, wherein the agent is an enzyme, an antibody, a primer nucleic acid, or a plasmid.

60. The method of any one of claims 54-56, wherein the agent is a cell.

61. The method of any one of claims 54-56, wherein the agent is a therapeutic agent or an imaging agent.

62. The method of any one of claims 27-61, further comprising adding a high molecular weight molecule in the liquid core.

63. The method of claim 62, wherein the high molecular weight molecule comprises at least one functional group for forming a linkage with an agent.

64. The method of claim 62 or 63, wherein the high molecular weight molecule is a high molecular weight polymer.

65. The method of any one of claims 27-64, further comprising adding a cross-linking molecule in the shell.

66. The capsule of claim 65, wherein the cross-linking molecule comprises at least one functional group for forming a linkage with an agent.

67. A method for preparing a capsule, the method comprising:
(i) forming an emulsion comprising droplets of an aqueous phase solution disposed in a non-aqueous phase solution, wherein the droplets comprise a liquid core surrounded by a shell comprising a poly(ethylene glycol)-poly(propylene glycol)-poly(ethylene glycol) diacrylate triblock copolymer; and
(ii) cross-linking the copolymer, and
wherein the capsule comprises: (a) a liquid core; and (b) a cross-linked shell surrounding the liquid core, wherein the cross-linked shell comprises a cross-linked poly(ethylene glycol)-poly(propylene glycol)-poly(ethylene glycol) triblock copolymer.

68. The method of claim 67, wherein the step of forming the emulsion comprises mixing the aqueous phase solution with the copolymer to form a first mixture and mixing the first mixture with the non-aqueous phase solution.

69. The method of claim 67 or 68, wherein the step of forming the emulsion is in a microfluidic device.

70. The method of claim 67, wherein the step of forming the emulsion comprises:
feeding the aqueous phase solution into a first microfluidic channel of a microfluidic device;
feeding the copolymer into a second microfluidic channel of the microfluidic device;
feeding the non-aqueous phase solution into a fourth microfluidic channel of the microfluidic device;
mixing the aqueous phase solution with the copolymer at a first intersection between the first microfluidic channel and the second microfluidic channel to form a first mixture;
conveying the first mixture through a third microfluidic channel toward a second intersection between the third microfluidic channel and the fourth microfluidic channel;
mixing the first mixture with the non-aqueous phase solution at the second intersection to form the emulsion; and
collecting the emulsion from the microfluidic device.

71. The method of any one of claims 67-70, wherein the cross-linking comprises photo-cross-linking.

72. The method of any one of claims 67-71, wherein the cross-linking comprises exposing the emulsion to ultraviolet (UV) radiation or visible light radiation.

73. The method of any one of claims 67-72, wherein the emulsion comprises a photoinitiator.

74. The method of claim 73, further comprising adding the photoinitiator to the emulsion prior to the step of cross-linking.

75. The method of claim 73 or 74, wherein the non-aqueous phase solution comprises a photoinitator.

76. The method of any one of claims 73-75, wherein the photoinitiator is a hydrophobic radical polymerization reaction photoinitiator.

77. The method of any one of claims 67-70, wherein the cross-linking comprises chemical cross-linking.

78. The method of any one of claims 67-77, wherein the non-aqueous phase solution comprises an oil.

79. The method of claim 78, wherein the oil is a fluorinated oil.

80. The method of claim 79, wherein the fluorinated oil is a fluorine-substituted alkylsiloxane or a fluorocarbon.

81. The method of any one of claims 67-80, wherein the cross-linked shell is semi-permeable.

82. The method of any one of claims 67-81, wherein the liquid core comprises a solute.

83. The method of claim 82, wherein the solute is a viscosity modifier.

84. The method of claim 82, wherein the solute is a sugar.

85. The method of claim 82, wherein the solute is dextran.

86. The method of claim 85, wherein the dextran has a molecular weight in the range of 3000 Daltons to 2,000,000 Daltons.

87. The method of any one of claims 67-86, wherein the capsule is a microcapsule.

88. The method of any one of claims 67-87, wherein the capsule has a diameter of 1 μm to 1000 μm.

89. The method of any one of claims 67-87, wherein the capsule has a diameter of 5 μm to 200 μm.

90. The method of any one of claims 67-89, wherein the capsule is optically clear.

91. The method of any one of claims 67-90, further comprising coating an outer surface of the cross-linked shell with a biocompatible material.

92. The method of any one of claims 67-91, further comprising adding an agent to the capsule.

93. The method of claim 92, wherein the agent is in the liquid core.

94. The method of claim 92, wherein the agent is in the cross-linked shell.

95. The method of claim 92-94, wherein the agent is a cell, biomolecule, polymer, small organic or inorganic molecule, microorganism or organoid.

96. The method of any one of claims 92-94, wherein the agent is an amino acid, peptide, polypeptide, nucleotide, oligonucleotide, polynucleotide, saccharide, oligosaccharide, or polysaccharide.

97. The method of claim 92-94, wherein the agent is an enzyme, an antibody, a primer nucleic acid, or a plasmid.

98. The method of claim 92-94, wherein the agent is a cell.

99. The method of claim 92-94, wherein the agent is a therapeutic agent or an imaging agent.

100. The method of any one of claims 67-99, further comprising adding a high molecular weight molecule to the liquid core.

101. The method of claim 100, wherein the high molecular weight molecule comprises at least one functional group for forming a linkage with an agent.

102. The method of claim 100 or 101, wherein the high molecular weight molecule is a high molecular weight polymer.

103. The method of any one of claims 67-102, further comprising adding a cross-linking molecule in the shell.

104. The method of claim 103, wherein the cross-linking molecule comprises at least one functional group for forming a linkage with an agent.

105. Use of a capsule of any one of claims 1-26 for single-cell genomics.

106. Use of a capsule of any one of claims 1-26 for immuno-isolation.

107. Use of a capsule of any one of claims 1-26 for cytometry.

108. Use of a capsule of any one of claims 1-26 for co-culture screen.

109. Use of a capsule of any one of claims 1-26 for micro-environmental screen.

110. Use of a capsule of any one of claims 1-26 for protein evolution.

111. Use of a capsule of any one of claims 1-26 for enzymatic cascade reaction.

112. Use of a capsule of any one of claims 1-26 for drug screening

113. Use of a capsule of any one of claims 1-26 for cell culture.

114. Use of a capsule of any one of claims 1-26 for polymerase chain reaction (PCR).

115. Use of a capsule of any one of claims 1-26 for imaging.

116. Use of a capsule of any one of claims 1-26 for drug delivery.

117. Use of a capsule of any one of claims 1-26 for diagnostic.

118. The capsule of any one of claims 14-16, wherein the agent is a microparticle or bead.

119. The capsule of claim 118, wherein the microparticle has a diameter of at least 100 nm.

120. The capsule of claim 118, wherein the microparticle has a diameter that is half the diameter of the capsule.

121. The capsule of claim 118, wherein the microparticle comprises a polymer.

122. The capsule of claim 118, wherein the microparticle comprises polystyrene or polymethacrylate.

123. The capsule of claim 118, wherein the bead has a diameter of at least 100 nm.

124. The capsule of claim 118, wherein the bead has a diameter that is half the diameter of the capsule.

125. The capsule of claim 118, wherein the bead comprises a polymer.

126. The capsule of claim 118, wherein the bead comprises polystyrene or polymethacrylate.

127. The capsule of any one of claims 1-22, further comprising a microparticle in the core.

128. The capsule of claim 127, wherein the microparticle comprises at least one functional group for forming a linkage with an agent.

129. The capsule of claim 127 or claim 128, wherein the microparticle is covalently linked or non-covalently linked to an antibody.

130. The capsule of claim 22 or claim 23, wherein the high molecular weight molecule is amino dextran, chitosan, or gelatin.

131. The capsule of claim 22 or claim 23, wherein the high molecular weight molecule is covalently linked or non-covalently linked to an antibody.

132. The method of claim 38, wherein the photoinitiator is dissolved in an organic solvent.

133. The method of claim 132, wherein the organic solvent containing the photoinitiator is dispersed into the non-aqueous phase.

134. The method of claim 133, wherein the non-aqueous phase comprises a hydrofluoroether oil.

135. The method of claim 132, wherein the organic solvent is hexane or acetone.

136. The method of claim 67, further comprising heating the emulsion to induce gelation of the shell phase before cross-linking the copolymer.

137. The method of claim 136, wherein the emulsion is heated to 20-50 °C.

138. The method of claim 136, wherein the emulsion is heated to 28 °C.
